(19) United States
(12) Patent Application Publication
(10) Pub. No.: US 2018/0116631 A1
(43) Pub. Date: May 3, 2018

(54) ULTRASOUND DIAGNOSTIC APPARATUS AND IMAGE FORMING METHOD

(71) Applicant: Konica Minolta, Inc., Tokyo (JP)

(72) Inventor: Tetsuya TANIGUCHI, Tokyo (JP)

(21) Appl. No.: 15/801,779

(22) Filed: Nov. 2, 2017

(30) Foreign Application Priority Data
Nov. 2, 2016 (JP) 2016-214792

(51) Int. Cl. A61B 8/14; A61B 8/08; A61B 8/00

(52) U.S. Cl. CPC A61B 8/14; A61B 8/4405; A61B 8/5215; A61B 8/5207

(57) ABSTRACT

An ultrasound diagnostic apparatus includes an ultrasound probe, a transmitter, a receiver, a signal intensity adjuster and an image data generator. The ultrasound probe transmits transmission ultrasound to an examination object, receives echo from the examination object and generates an echo signal. The transmitter generates a drive signal and outputs the drive signal to the ultrasound probe to cause the ultrasound probe to generate the transmission ultrasound. The receiver receives the echo signal from the ultrasound probe. The signal intensity adjuster adjusts signal intensity of the echo signal to signal intensity having a flat frequency range. The image data generator generates ultrasound image data from the echo signal having the adjusted signal intensity.

### CROSS-REFERENCE TO RELATED APPLICATIONS

The present invention claims priority under 35 U.S.C. § 119 to Japanese Patent Application 2016-214792, filed Nov. 2, 2016, the entire contents of which being incorporated herein by reference.

### BACKGROUND

### 1. Technological Field

The present invention relates to an ultrasound diagnostic apparatus and an image forming method.

### 2. Description of the Related Art

Ultrasound images used for ultrasound diagnosis are generated from, as electric echo signals corresponding to ultrasound (echo) received from an examination object(s) to which ultrasound (transmission ultrasound) has been transmitted, (i) reflected echo signals obtained by the transmission ultrasound being reflected by, in the examination object, structures larger than the wavelength of the ultrasound and (ii) scattered echo signals obtained by the transmission ultrasound being scattered by, in the examination object, structures smaller than the wavelength of the transmission ultrasound, in general.

The reflected echo signal(s) is a signal of reached ultrasound, which has reached a structure, being reflected by its tissue interface at an intensity corresponding to a difference in acoustic impedance, thereby being ultrasound having the original form or the positive-negative reversed form, so that information on the interface can be obtained directly at a resolution corresponding to the reached ultrasound. However, in a living body, structures smaller than the wavelength of reached ultrasound are also present, and if such structures exist with a distance therebetween shorter than the wavelength, the scattered echo signals generated thereby interfere, so that the forms are different from the waveform of the reached ultrasound. Thus, the scattered echo signals do not mirror the shapes of the structures directly.

However, the scattered echo signals are results of scattering/interference derived from tissue, and are observed as the so-called speckles at substance parts, such as the liver and the thyroid gland, and their uniformity, granularity and so forth are utilized as parts of diagnosis information. There have been conceived, for example, a method for determining the stage of progression of hepatic cirrhosis and a method for extracting/observing microstructures by making use of statistical nature of speckles, which have been used by operators' subjective views only. (Refer to Japanese Patent Application Publication No. 2011-224410.)

As another method for extracting microstructures, there has been known a method of separating a microstructure from a continuous structure by making use of spatial continuity, thereby extracting the microstructure. (Refer to Japanese Patent Application Publication No. 2013-56178.)

As another method therefor, there has been proposed a method of generating multiple types of image data based on intensity change of multiple frequency components, performing spatial filtering on at least one type of the image data, and combining these image data, so as to obtain effects, such as enhancement of specific tissues. (Refer to Japanese Patent Application Publication No. 2006-204594.)

Although the method described in Japanese Patent Application Publication No. 2011-224410 can extract the position of a microstructure, it cannot solve the problem that the structure of a microstructure cannot be recognized because image information there is the same as the conventional one.

Further, because the method described in Japanese Patent Application Publication No. 2013-56178 is a method for determining, by spatial spread, whether a high brightness part is derived from a microstructure, it cannot solve the problem that the structure of a microstructure cannot be recognized.

Further, although the method described in Japanese Patent Application Publication No. 2006-204594 is effective in enhancement of specific tissues by making use of difference in reflection frequency characteristics of tissues, it forms images by making the original information pass through multiple bandpass filters to limit the band, so that the resolution in the distance direction decreases. In addition, because the respective pieces of the image information are based on envelope-detected signals having no phase information, even if they are superimposed, a band widening effect based on the so-called waveform superimposing principle cannot be obtained. Thus, although the method may obtain an image smoothing effect, it does not increase information density and hence does not contribute to improvement of delineation of the structure of a microstructure. Further, as to delineation of speckles, although there is described reduction of speckles, there is not described or suggested fine granulation of speckles at all.

### SUMMARY

Objects of the present invention include finely granulating speckles as small/fine as possible, the speckles being generated by interference of scattered echo signals derived from scattering substances, and extracting microstructures with the speckles as the minimum units of significant image information.

In order to achieve at least one of the abovementioned objects, according to an aspect of the present invention, there is provided an ultrasound diagnostic apparatus including: an ultrasound probe that transmits transmission ultrasound to an examination object, receives echo from the examination object and generates an echo signal; a transmitter that generates a drive signal and outputs the drive signal to the ultrasound probe to cause the ultrasound probe to generate the transmission ultrasound; a receiver that receives the echo signal from the ultrasound probe; a signal intensity adjuster that adjusts signal intensity of the echo signal to signal intensity having a flat frequency range; and an image data generator that generates ultrasound image data from the echo signal having the adjusted signal intensity.

In order to achieve at least one of the abovementioned objects, according to another aspect of the present invention, there is provided an image forming method including: adjusting signal intensity of an echo signal received from an ultrasound probe that transmits transmission ultrasound to an examination object and receives echo from the examination object to signal intensity having a flat frequency range; and generating ultrasound image data from the echo signal having the adjusted signal intensity.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention, wherein:

FIG. 1 is an external view of an ultrasound diagnostic apparatus according to one or more embodiments of the present invention;

FIG. 2 is a block diagram showing the functional configuration of the ultrasound diagnostic apparatus;

FIG. 3 is a block diagram showing the functional configuration of a transmitter;

FIG. 4A shows frequency characteristics of signal intensity of transmission ultrasound in Triad-THI, which is the broadband transmission/reception method described in Japanese Patent Application Publication Nos. 2014-168555 and 2016-214622;

FIG. 4B shows frequency characteristics of echo when depth of Triad-THI is at or near the focal point;

FIG. 5 shows frequency characteristics of signal intensity of an echo signal;

FIG. 6A shows frequency characteristics of signal passing degree of a signal intensity correction filter of the embodiment(s);

FIG. 6B shows frequency characteristics of signal intensity of a first imaging signal obtained by filtering with the signal intensity correction filter of the embodiment(s) in relation to the highest signal intensity of the first imaging signal being 0 dB;

FIG. 7A shows time waveform characteristics of signal intensity of the first imaging signal;

FIG. 7B is a conceptual diagram schematically showing speckle granularity characteristics of the first imaging signal;

FIG. 8A shows frequency characteristics of signal passing degree of a conventional signal intensity correction filter;

FIG. 8B shows frequency characteristics of signal intensity of a second imaging signal obtained by filtering with the conventional signal intensity correction filter in relation to the highest signal intensity of the second imaging signal being 0 dB;

FIG. 9A shows time waveform characteristics of signal intensity of the second imaging signal;

FIG. 9B is a conceptual diagram schematically showing speckle granularity characteristics of the second imaging signal;

FIG. 10 shows frequency characteristics of normalized sensitivity of a first ultrasound probe;

FIG. 11A shows time characteristics of signal intensity of a first drive signal;

FIG. 11B shows power spectrum of the first drive signal;

FIG. 12A shows time characteristics of signal intensity of first transmission ultrasound;

FIG. 12B shows power spectrum of the first transmission ultrasound;

FIG. 13 shows filter characteristics of a first signal intensity correction filter;

FIG. 14 shows filter characteristics of a second signal intensity correction filter;

FIG. 15 shows filter characteristics of a third signal intensity correction filter;

FIG. 16 shows filter characteristics of a fourth signal intensity correction filter;

FIG. 17A shows time characteristics of signal intensity of a second drive signal;

FIG. 17B shows power spectrum of the second drive signal;

FIG. 18A shows time characteristics of signal intensity of second transmission ultrasound;

FIG. 18B shows power spectrum of the second transmission ultrasound;

FIG. 19 shows filter characteristics of a sixth signal intensity correction filter;

FIG. 20 shows frequency characteristics of normalized sensitivity of a second ultrasound probe;

FIG. 21A shows time characteristics of signal intensity of a third drive signal;

FIG. 21B shows power spectrum of the third drive signal;

FIG. 22A shows time characteristics of signal intensity of third transmission ultrasound;

FIG. 22B shows power spectrum of the third transmission ultrasound;

FIG. 23 shows filter characteristics of a seventh signal intensity correction filter;

FIG. 24 shows filter characteristics of an eighth signal intensity correction filter;

FIG. 25A shows a B-mode image;

FIG. 25B shows an image obtained by watershed segmentation on the B-mode image;

FIG. 26A shows wavelength characteristics of echo intensity of a combined echo signal of a first echo signal and a second echo signal; and

FIG. 26B shows wavelength characteristics of echo intensity of the absolute value and the mean values in divided wavelength of the combined echo signal.

### DETAILED DESCRIPTION OF EMBODIMENTS

Hereinafter, one or more embodiments of the present invention will be described with reference to the drawings. However, the scope of the invention is not limited to the disclosed embodiments. In the following explanation, the same reference number(s) is given to those having the same function and configuration, and expiation thereof is not repeated.

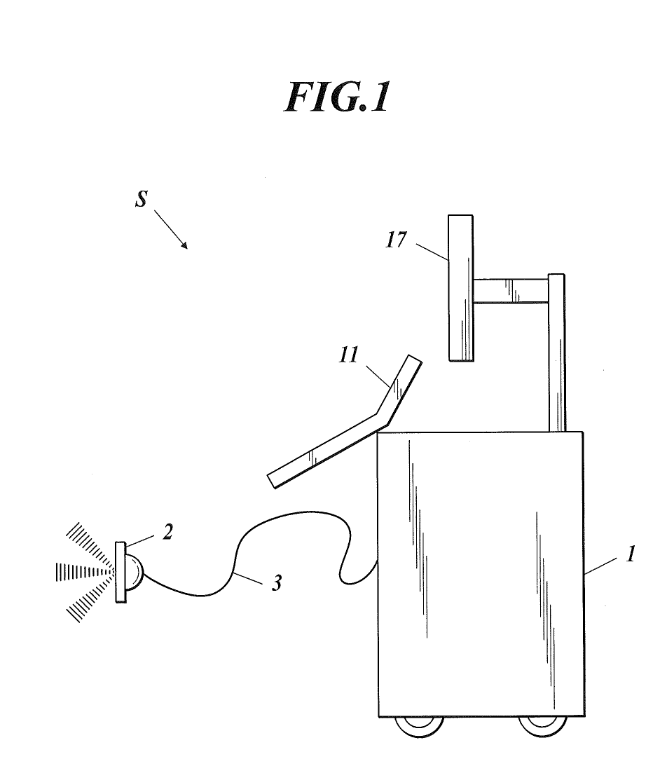

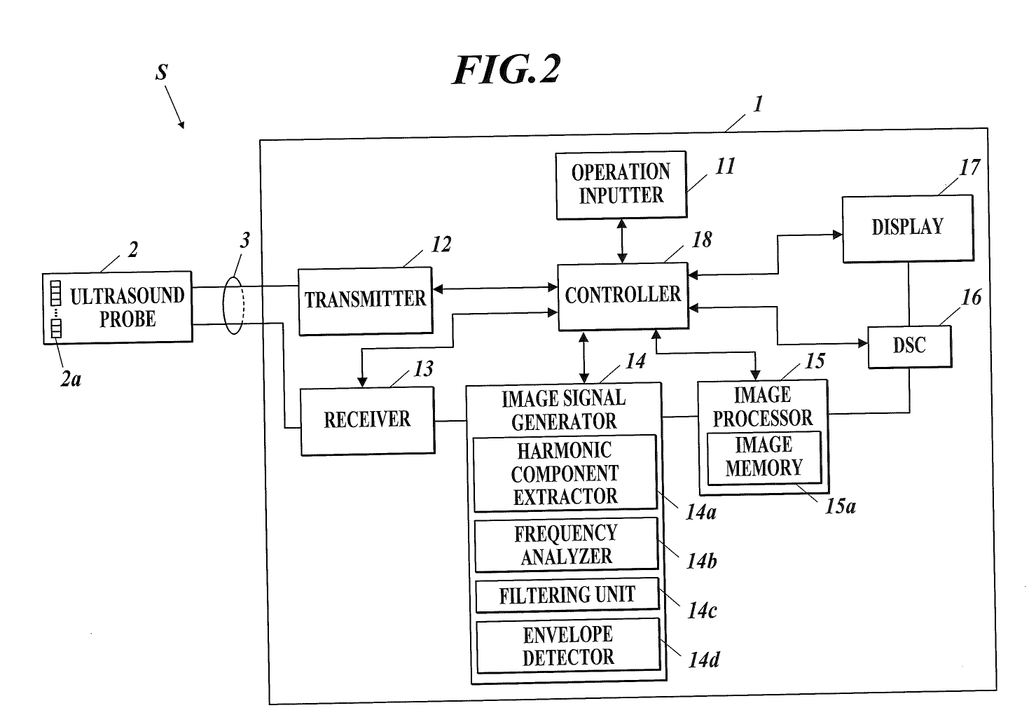

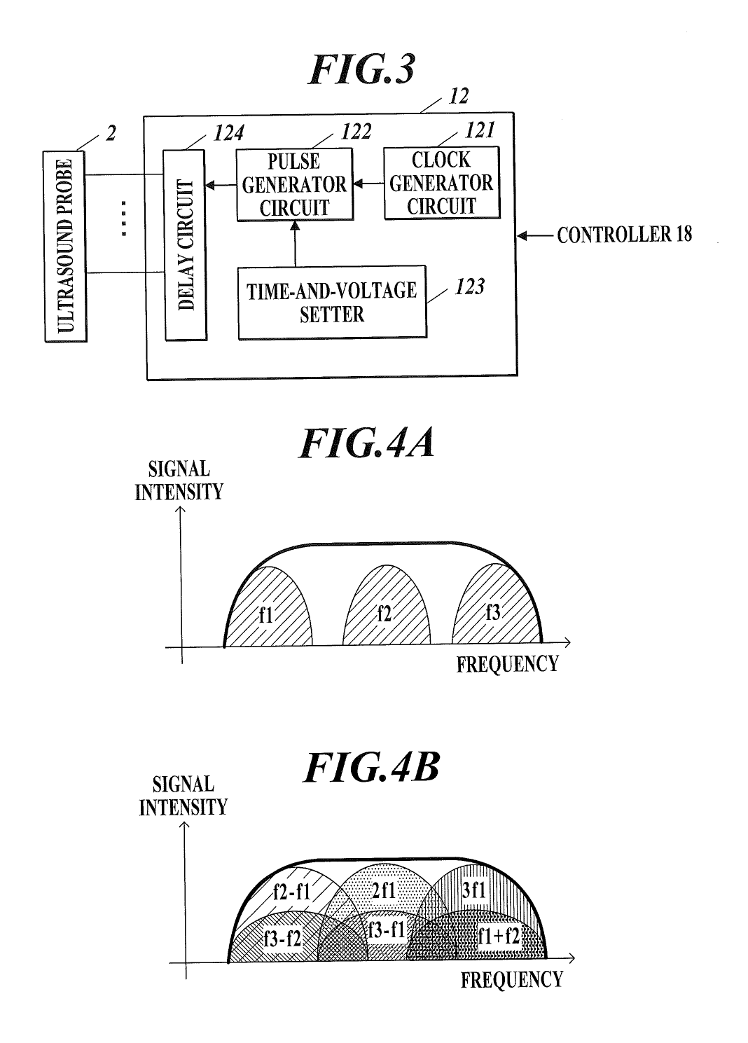

First, with reference to FIG. 1 to FIG. 3, the configuration of an ultrasound diagnostic apparatus S according to one or more embodiments of the present invention is described. FIG. 1 is an external view of the ultrasound diagnostic apparatus S according to the embodiment, FIG. 2 is a block diagram showing the functional configuration of the ultrasound diagnostic apparatus S, and FIG. 3 is a block diagram showing the functional configuration of a transmitter 12.

The ultrasound diagnostic apparatus S generates ultrasound images by a method that can delineate microstructures in scattering regions, the method being different from the conventional approach of receiving and imaging reflected echo signal(s) with a single peak frequency band shape represented by Gaussian approximation, thereby obtaining a Gaussian-approximated envelope shape of the reflected echo signal. More specifically, the ultrasound diagnostic apparatus S daringly flattens a frequency band shape of an echo signal(s) received in a wide band, thereby making the shape have no peak substantially. This diversifies the scattering/interference pitch at the ultrasound waveform level, namely, maximizes the multiple interference effect and finely granulates speckles, and thereby can delineate microstructures of the scattering level with the speckles.

As shown in FIG. 1 and FIG. 2, the ultrasound diagnostic apparatus S includes a main part 1 of the ultrasound diagnostic apparatus S and an ultrasound probe 2. The ultrasound probe 2 transmits ultrasound(s) (transmission ultrasound(s)) to not-shown examination objects of living bodies or the like, and receives ultrasound(s) (echo(es)) reflected or scattered by the examination objects. The main part 1 is connected to the ultrasound probe 2 through a cable 3, and transmits electric drive signals to the ultrasound probe 2 to cause the ultrasound probe 2 to transmit transmission ultrasound to examination objects, and images the internal states of the examination objects as ultrasound image data on the basis of electric echo signals generated by the ultrasound probe 2 according to echo received from the examination objects.

The ultrasound probe 2 includes transducers 2a composed of piezoelectric elements and an acoustic lens (not shown) that focuses transmission ultrasound on the focal point. The transducers 2a are arranged, for example, in a one-dimensional array in the orientation (azimuth) direction. In the embodiment, the ultrasound probe 2 has, for example, 192 transducers 2a. The transducers 2a may be arranged in a two-dimensional array. The number of transducers 2a can be arbitrarily set. In the embodiment, the ultrasound probe 2 is an electronic scanning probe employing linear scanning, but may be one employing either electric scanning or mechanical scanning, or one employing one of linear scanning, sector scanning and convex scanning.

The shape and the center frequency of the ultrasound probe 2 used in the embodiment are not particularly limited, but as its frequency band characteristics, a transmission/reception −20 dB fractional bandwidth of 120% or more is preferable. The “transmission/reception −20 dB fractional bandwidth” is a value obtained, using an upper-end frequency FH20 and a lower-end frequency FL20 at a normalized sensitivity of −20 dB in frequency characteristics of normalized sensitivity of the ultrasound probe 2, by dividing their difference (FH20−FL20) by their center frequency ((FH20−FL20)/2)). If the −20 dB fractional bandwidth is narrow, even when echo signal intensity is flattened, the interference pitch is not sufficiently diversified, and the effect of finely granulating speckles is not sufficient.

Note that communication between the main part 1 and the ultrasound probe 2 may be wireless communication, such as UWB (Ultra WideBand) communication, instead of wire communication through the cable 3.

As shown in FIG. 2, the main part 1 includes, for example, an operation inputter 11, a transmitter 12, a receiver 13, an image signal generator 14, an image processer 15, a DSC (Digital Scan Converter) 16, a display 17 and a controller 18 as a display controller.

The operation inputter 101 includes, for example, various switches, buttons, a trackball, a mouse and a keyboard to input, for example, commands to start diagnosis and data such as personal information about examination objects, and outputs operation signals to the controller 18.

The transmitter 12 is a circuit that supplies electric drive signals to the ultrasound probe 2 through the cable 3 under the control of the controller 18 to cause the ultrasound probe 2 to generate transmission ultrasound. More specifically, as shown in FIG. 3, the transmitter 12 includes, for example, a clock generator circuit 121, a pulse generator circuit 122, a time-and-voltage setter 123 and a delay circuit 124.

The clock generator circuit 121 generates clock signals that determine transmission timing and transmission frequency of drive signals. The pulse generator circuit 122 generates pulse signals as drive signals at predetermined intervals. The pulse generator circuit 122 switches, for example, voltages of three levels (+HV, 0(GND), −HV) or five levels (+HV, +MV, 0(GND), −MV, −HV) to output, thereby generating a square-wave drive signal(s). Here, the positive amplitude and the negative amplitude of the pulse signal are the same, but this is not a limit. Further, in the embodiment, voltages of three or five levels are switched to output a drive signal(s), but the number of levels thereof is not limited to three or five. The number thereof may be arbitrarily set, but preferably five or less. This can improve degree of freedom in control of frequency components at low cost, and can generate transmission ultrasound for higher resolution.

The time-and-voltage setter 123 sets the duration and the voltage level of each section at the same voltage of drive signals output from the pulse generator circuit 122. That is, the pulse generator circuit 122 outputs a drive signal the pulse waveform of which accords with the duration and the voltage level of each section set by the time-and-voltage setter 123. The duration and the voltage level of each section set by the time-and-voltage setter 123 can be changed, for example, by input operations with the operation inputter 11.

The delay circuit 124 sets delay time for transmission timing of a drive signal for each path corresponding to each transducer 2a, and delays transmission of the drive signals by the set delay times to converge transmission beams of transmission ultrasound.

Thus-configured transmitter 12 successively shifts, under the control of the controller 18, the transducers 2a, to each of which the transmitter 12 supplies the drive signal, by a predetermined number of the transducers 2a each time ultrasound is transmitted/received, thereby supplying the drive signal to each of the selected transducers 2a. Thus, scanning is performed.

As the method for transmitting/receiving ultrasound in the embodiment, a method which can obtain an echo signal(s) the intensity of which can be adjusted to a frequency range to be flattened (hereinafter may be called the “flattening frequency range” or “flat frequency range”) is chosen. For example, if fundamentals are imaged, transmission that covers a frequency band wider than the flattening frequency range is necessary. In the embodiment, for example, THI (Tissue Harmonic Imaging) can be performed. THI is a method for generating ultrasound images by using, harmonic components, from transmission ultrasound, of echo signals. In the case of THI, the transmission frequency band is not limited, but transmission/reception to make the frequency band of harmonic signals generated from the transmission fundamental components by nonlinear propagation wider than the frequency band (frequency range) to be flattened is necessary. Between fundamental imaging and THI, THI, which has sound pressure dependency in the generating and can reduce sidelobes and exhibit a beam sharpening effect in the slice direction, is preferable.

In the embodiment, in order to extract harmonic components used in THI, pulse inversion can be performed. That is, when pulse inversion is performed, the transmitter 12 can transmit, as the drive signal(s), a first pulse signal and a second pulse signal having a reverse polarity to that of the first pulse signal onto the same scanning line(s) with a time interval in between. The second pulse signal may be one that is time-reversed from the first pulse signal. Alternatively, the method utilizing an unadded remainder component of the first pulse signal and the second pulse signal that are asymmetric described in Japanese Patent No. 5924296 may be used.

Further, in the embodiment, as THI, Triad-THI, which is described in Japanese Patent Application Publication Nos. 2014-168555 and 2016-214622 as the broadband transmission/reception method, can be performed. Triad-THI is a method of outputting transmission ultrasound composed of fundamentals of three frequency components and generating ultrasound images with harmonic components of an echo signal(s) based on received echo. That is, the transmitter 12 generates a drive signal having fundamental components of three frequency components when Triad-THI is performed. Thus, the transmitter 12 can generate drive signals for Triad-THI and for pulse inversion.

The receiver 13 is a circuit that receives electric echo signals from the ultrasound probe 2 through the cable 3 under the control of the controller 18. The receiver 13 includes, for example, an amplifier, an A/D converter circuit and a phasing adding circuit. The amplifier is a circuit that amplifies an echo signal at an amplification factor preset for each path corresponding to each transducer 2a. The A/D converter circuit performs analog-digital conversion (A/D conversion) on the amplified echo signals. The phasing adding circuit gives delay time for each path corresponding to each transducer 2a to the A/D converted echo signal to adjust time phase(s) and add up (phase-add) the echo signals to generate sound ray data.

Here, with reference to FIG. 4A and FIG. 4B, ultrasound transmission/reception in Triad-THI is described. FIG. 4A shows frequency characteristics of signal intensity of transmission ultrasound in Triad-THI, which is the broadband transmission/reception method described in Japanese Patent Application Publication Nos. 2014-168555 and 2016-214622. FIG. 4B shows frequency characteristics of echo when depth of Triad-THI is at or near the focal point.

When Triad-THI is performed, the transmitter 12 generates, as shown in FIG. 4A as an example, a drive signal for causing the ultrasound probe 2 to output transmission ultrasound containing fundamentals f1, f2 and f3. FIG. 4A shows frequency on the horizontal axis, sensitivity (signal intensity) on the vertical axis, and frequency component(s) (transmission/reception frequency band) of the ultrasound probe 2 with a bold solid line.

More specifically, for example, time waveforms of three signals of frequencies corresponding to the fundamentals f1, f2 and f3 in the transmission/reception frequency band of the ultrasound probe 2 are subjected to at least one of AM (Amplitude Modulation) and FM (Frequency Modulation) and filtered with time window, such as Hanning window or rectangular window (do-nothing window), the obtained three time waveforms are multiplied by their respective appropriate magnification factors and then summed up, and a bias in the amplitude direction not affecting transmission of ultrasound is added to the whole waveform. The transmitter 12 generates a drive signal having a time waveform obtained by allotting the waveform of the bias-added signal to voltage values of five levels or so.

The echo signal from the focal point or near there corresponding to the transmission ultrasound shown in FIG. 4A has a feature shown in FIG. 4B. FIG. 4B shows frequency on the horizontal axis, signal intensity on the vertical axis, frequency components composed of frequency components of echo with medium solid lines, and frequency component(s) (transmission/reception frequency band) of the ultrasound probe 2 with a bold solid line. The echo signal obtained contains the harmonic components (f2−f1, 2f1, 3f1, f3−f2, f3−f1, f1+f2) shown in FIG. 4B in the transmission/reception frequency band of the ultrasound probe 2. Thus, the receiver 12 receives echo containing harmonic components and generates an electric echo signal(s) (sound ray data) from the echo.

The image signal generator 14, under the control of the controller 18, performs envelope detection, logarithmic amplification or the like on the echo signal (sound ray data) from the receiver 13 and adjusts gain or the like to perform brightness conversion, thereby generating an image signal of an B-mode image (B-mode image data). That is, the B-mode image data expresses intensity of the echo signal by brightness. The B-mode image data generated by the image signal generator 14 is sent to the image processor 15. The image signal generator 14 includes a harmonic component extractor 14a, a frequency analyzer 14b, a filtering unit 14c as a signal intensity adjuster, and an envelope detector 14d as an image data generator.

The harmonic component extractor 14a extracts harmonic components from the echo signal (sound ray data) output from the receiver 13 by pulse inversion and outputs an echo signal composed of the harmonic components, under the control of the controller 18. The harmonic components can be extracted as follows: add up (combine) echo signals obtained from echoes respectively corresponding to two transmission ultrasounds respectively generated from the above-described first pulse signal and second pulse signal; remove fundamental components contained in the combined echo signal; and then extract the harmonic components.

The frequency analyzer 14b performs frequency analysis on the echo signal (sound ray data) of the harmonic components (determination of intensities of the respective frequency components in the flattening frequency range) extracted by the harmonic component extractor 14a and outputs the analysis result to the filtering unit 14c, under the control of the controller 18. The flattening frequency range is set such that the range is in an imaging frequency range and the ratio of an upper-end frequency to a lower-end frequency (hereinafter may be called the “upper-end-frequency-to-lower-end-frequency ratio) of the range is 2.0 or more. The value may be a fixed value set by a designer or a value changeable by an operator. Even if the value is a value changeable by an operator, its changeable range is set to meet the above setting conditions. If the value is a value changeable by an operator, the operator needs to operate the value according to an observation site or a purpose of observation, considering that as the flattening frequency range is wider, the effect of finely granulating speckles is higher, but the S/N (signal-to-noise ratio) is lower.

The filtering unit 14c filters the sound ray data of the harmonic components extracted by the harmonic component extractor 14a with a signal intensity correction filter, which is in charge of flattening signal intensity characteristics, and outputs the filtered echo signal (an imaging signal), under the control of the controller 18.

Flattening of the frequency of an echo signal with a signal intensity correction filter may be perform on the echo signal immediately after being received, namely, on the echo signal before being phase-added in the receiver 13, and may be at the analog signal level before being A/D converted or at the digital signal level (sound ray data) after being A/D converted in the receiver 13, but preferably on the echo signal at the digital signal level after being phase-added (echo signal of harmonic components) with a digital filter in the image signal generator 14 because it can simplify the apparatus.

The digital filter as a signal intensity correction filter may be any conventional one, such as FIR (Finite Impulse Response) or IIR (Infinite Impulse Response), without limits. However, when consideration is given to influence on the phases, FIR is preferable. Further, the method of connecting two or more FIR filters in series described in Japanese Patent Application Publication No. 2003-19135 is preferable because this allows separate and individual coefficients to be set to a noise cutting filter(s), such as a high-pass filter and/or a low-pass filter, and a signal intensity correction filter, which is in charge of flattening signal intensity characteristics, and makes them easy to control signal flattening and to handle the below-described adaptive processing.

The signal intensity correction filter used in the filtering unit 14c may be a non-adaptive signal intensity correction filter, the coefficient of which is predetermined for each depth from depth change characteristics of signal intensity of harmonic components generated from transmission ultrasound and/or reception sensitivity characteristics of the ultrasound probe 2. Use of such a filter can exhibit the effect(s) at a sufficient level. However, frequency characteristics of scattered echo are somewhat different from an observation site to an observation site. Then, a method for adaptively changing the coefficient of a signal intensity correction filter can always exhibit the effect(s) at the best level, regardless of observation sites. This adaptive processing is performed by use of an adaptive signal intensity correction filter.

When using an adaptive signal intensity correction filter, the filtering unit 14c sets the coefficient of the signal intensity correction filter according to the value of intensity of each frequency component in the flattening frequency range. More specifically, the frequency analyzer 14b obtains the frequency spectrum of the echo signal (sound ray data) in the region of interest (ROI) by FFT (Fast Fourier Transform) analysis, and the filtering unit 14c sets the coefficient of the signal intensity correction filter in such a way as to offset the frequency intensity distribution in the flattening frequency range obtained by FFT analysis.

The frequency analysis in the frequency analyzer 14b and updating of the coefficient of the signal intensity correction filter in the filtering unit 14c may be performed for each frame, for every several frames or at predetermined time intervals.

Thus, when using an adaptive signal intensity correction filter, the filtering unit 14c sets the coefficient of the adaptive signal intensity correction filter according to the analysis result of the frequency analysis output from the frequency analyzer 14b. On the other hand, when using a non-adaptive signal intensity correction filter, the filtering unit 14c uses a non-adaptive signal intensity correction filter, the coefficient of which is preset.

The envelope detector 14d performs envelope detection, logarithmic amplification or the like on the imaging signal output from the filtering unit 14c and adjusts gain or the like to perform brightness conversion, thereby generating an image signal of a B-mode image (B-mode image data).

The image processor 15 includes an image memory 15a composed of a semiconductor memory, such as a DRAM (Dynamic Random Access Memory). The image processer 15 stores the B-mode image data output from the image signal generator 14 in the image memory 15a in units of frames under the control of the controller 18. The image data in units of frames may be referred to as ultrasound image data or frame image data. The image processor 15 reads the ultrasound image data from the image memory 15a and outputs the ultrasound image data to the DSC 16 timely.

The DSC 16 performs coordinate conversion or the like on the ultrasound image data received from the image processor 15, thereby converting the ultrasound image data into image signals for display, and outputs the image signals to the display 17, under the control of the controller 18.

As the display 17, an LCD (Liquid Crystal Display), a CRT (Cathode Ray Tube) display, an organic EL (Electronic Luminescence) display, an inorganic EL display, a plasma display or the like can be used. The display 17 displays ultrasound images on its display screen in response to the image signals output from the DSC 16.

The controller 18 can be realized by a hardware processor, and includes, for example, a CPU (Central Processing Unit), a ROM (Read Only Memory) and a RAM (Random Access Memory), and reads a system program and various process programs stored in the ROM, opens the read programs on the RAM and performs centralized control of actions of the devices or the like of the ultrasound diagnostic apparatus S in accordance with the opened programs. The ROM is composed of a nonvolatile memory, such as a semiconductor memory, and stores therein, for example, the system program for the ultrasound diagnostic apparatus S; the various process programs executable on the system program; and various data. These programs are stored in the form of computer readable program codes, and the CPU acts, following the program codes. The RAM provides a work area where the various programs to be executed by the CPU and data relevant to the programs are temporarily stored.

As to the devices or the like (the transmitter 12, the receiver 13, the harmonic component extractor 14a, the frequency analyzer 14b, the filtering unit 14c, the envelope detector 14d, etc.) of the main part 1, all or some of the functions of the respective function blocks can be realized by a hardware circuit(s), such as an integrated circuit(s). The integrated circuit is, for example, an LSI (Large Scale Integration), and LSI may be called an IC, a system LSI, a super LSI or an ultra LSI, depending on the degree of integration. The method for forming the integrated circuit is not limited to LSI, and hence the functions may be realized by a dedicated circuit or a versatile processor, or realized by making use of an FPGA (Field Programmable Gate Array) or a reconfigurable processor that can reconfigure connection and setting of circuit cells in LSI. Alternatively, all or some of the functions of the respective function blocks may be performed by software. In this case, the software is stored in one or more of storage media, such as ROMs, optical disks and hard disks, and performed by an arithmetic logic unit.

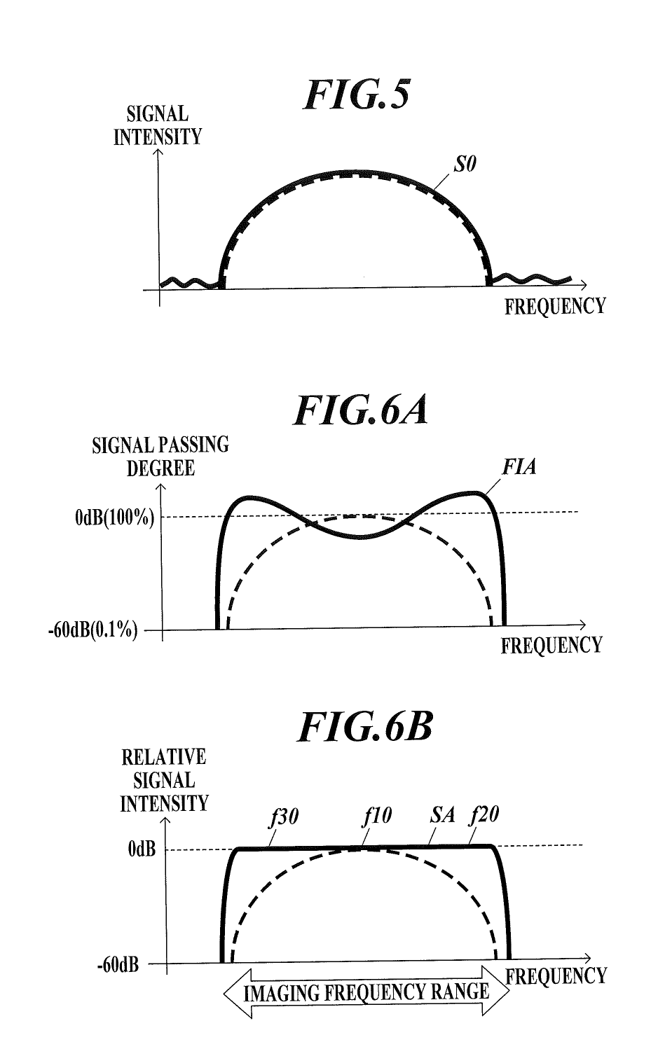

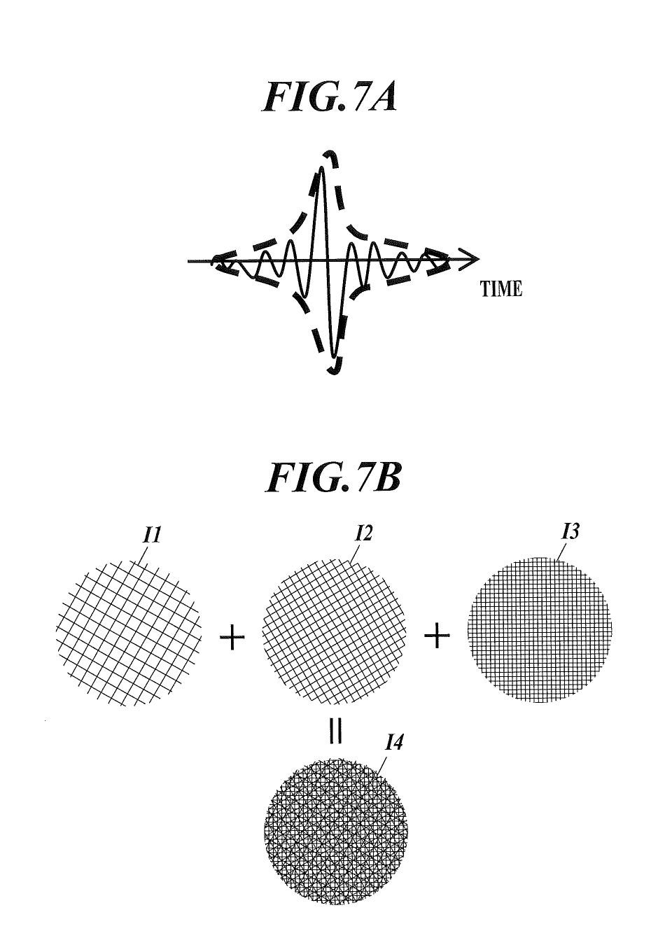

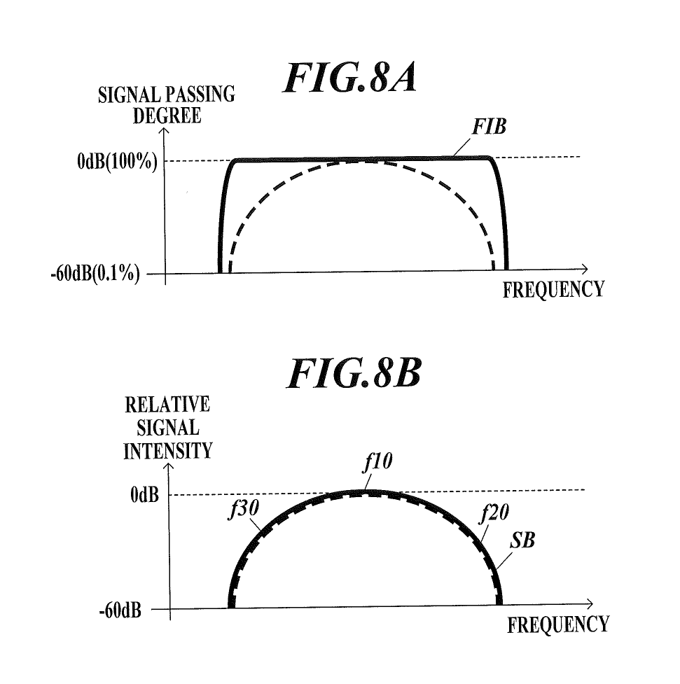

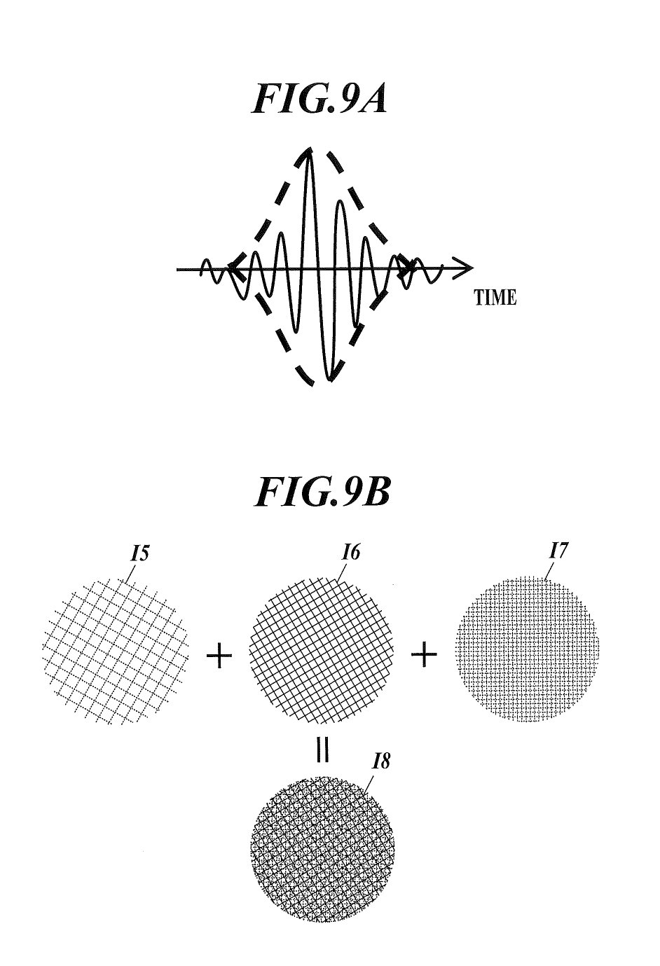

Next, with reference to FIG. 5 to FIG. 9B, filtering in the filtering unit 14c is described. FIG. 5 shows frequency characteristics of signal intensity of an echo signal S0. FIG. 6A shows frequency characteristics of signal passing degree (dB) of a signal intensity correction filter FIA according to the embodiment, wherein the dotted line shows 0 dB, namely, a transmittance of 100%, and the bottom of the vertical axis shows −60 dB, namely, a transmittance of 0.1%. In a frequency range where the signal passing degree is larger than 0 dB, the signal is amplified. FIG. 6B shows frequency characteristics of signal intensity of an imaging signal SA obtained by filtering with the signal intensity correction filter FIA in relation to the highest signal intensity of the imaging signal SA being 0 dB, wherein the vertical axis is expressed by dB as with FIG. 6A. FIG. 7A shows time waveform characteristics of the imaging signal SA. FIG. 7B is a conceptual diagram schematically showing speckle granularity characteristics of the imaging signal SA. FIG. 8A shows frequency characteristics of signal passing degree (dB) of a conventional signal intensity correction filter FIB. FIG. 8B shows, as with FIG. 6B, frequency characteristics of signal intensity of an imaging signal SB obtained by filtering with the signal intensity correction filter FIB in relation to the highest signal intensity of the imaging signal SB being 0 dB. FIG. 9A shows time waveform characteristics of the imaging signal SB. FIG. 9B is a conceptual diagram schematically showing speckle granularity characteristics of the imaging signal SB.

For example, in the ultrasound diagnostic apparatus S, the ultrasound probe 2 outputs transmission ultrasound, the receiver 13 receives echo through the ultrasounds probe 2 and generates an echo signal (sound ray data), and the harmonic component extractor 14a generates an echo signal (sound ray data) S0, which is composed of harmonic components and has the characteristics shown in FIG. 5. FIG. 5 shows frequency on the horizontal axis, signal intensity on the vertical axis, signal intensity of the echo signal S0 with a solid line, and a frequency band of the ultrasound probe 2 with a broken line. With the normal THI, it is difficult to obtain a reception signal(s) that thoroughly satisfies the whole frequency band of an ultrasound probe. However, for example, if the broadband transmission/reception method described in Japanese Patent Application Publication Nos. 2014-168555 and 2016-214622 is used, as shown in FIG. 5, a reception signal, namely, the echo signal S0, the signal intensity of which thoroughly satisfies the inside of the frequency band of the ultrasound probe 2, can be obtained.

Then, the filtering unit 14a filters the echo signal S0 with the signal intensity correction filter FIA having the characteristics shown in FIG. 6A. FIG. 6A shows frequency on the horizontal axis, signal passing degree on the vertical axis, filter characteristics of the signal intensity correction filter FIA with a solid line, and a frequency band of the ultrasound probe 2 with a broken line. The same applies to FIG. 8A. The signal intensity correction filter FIA has a high signal passing degree(s) at a part(s) where the signal intensity of the echo signal S0 is low and has a low signal passing degree(s) at a part(s) where the signal intensity of the echo signal S0 is high, in a range containing the frequency band of the ultrasound probe 2.

The echo signal filtered in the filtering unit 14c is the imaging signal SA having the characteristics shown in FIG. 6B. FIG. 6B shows frequency on the horizontal axis, signal intensity on the vertical axis, signal intensity of the imaging signal SA with a solid line, and a frequency band of the ultrasound probe 2 with a broken line. The same applies to FIG. 8B. The imaging signal SA has characteristics having a flat frequency range (i.e. the flattening frequency range that has been flattened in the filtering unit 14c) in the range containing the frequency band of the ultrasound probe 2.

In the imaging signal SA, the center frequency of the imaging frequency range is represented by a frequency f10, a predetermined frequency higher than the frequency f10 is represented by a frequency f20, and a predetermined frequency lower than the frequency f10 is represented by a frequency f30. The imaging frequency range of the embodiment is a range of continuous frequencies where the sensitivity is not below −40 dB from the highest sensitivity (the highest dB value) in a characteristic curve obtained by adding up reception sensitivity characteristics of an ultrasound probe, the highest sensitivity of which is normalized by 0 dB, and filter characteristics of a signal intensity correction filter.

The imaging signal SA has time characteristics shown in FIG. 7A. FIG. 7A shows time on the horizontal axis, signal intensity in the vertical direction, signal intensity of the imaging signal SA with a solid line, and an envelope of the imaging signal SA with a broken line. The same applies to FIG. 9A.

FIG. 7B is a conceptual diagram of delineation of a scattering tissue at the frequencies f10, f20 and f30 of the imaging signal SA. Interference pattern images of the scattering tissue corresponding to the frequencies f10, f20 and f30 are mesh images I1, I2 and I3, respectively. In each of the images I1, I2 and I3, the signal intensity of the imaging signal SA is expressed by continuity of the lines of the mesh. As more solid lines are dotted, which indicates broken parts, the signal intensity of the imaging signal SA is weaker (lower). Further, in each of the images I1, I2 and I3, the size of the interference pattern at the frequency (wavelength) of the imaging signal SA is expressed by spaces between the lines of the mesh. As the spaces between the lines are larger, the repeating unit of the imaging signal SA is larger, and the speckles are larger and coarser. An image I4 is formed by combining the images I1, I2 and I3.

Similarity, in a conventional ultrasound diagnostic apparatus, the echo signal S0 of the harmonic components having the characteristics shown in FIG. 5 is generated, and a filtering unit thereof uses the signal intensity correction filter FIB having the characteristics shown in FIG. 8A. The signal intensity correction filter FIB has a constant signal passing degree of 0 dB (a transmittance of 100%) in the range containing the frequency band of the ultrasound probe 2.

The echo signal filtered in the filtering unit of the conventional ultrasound diagnostic apparatus is the imaging signal SB having the characteristics shown in FIG. 8B. The imaging signal SB has time characteristics shown in FIG. 9A. FIG. 9B is a conceptual diagram of delineation of a scattering tissue at the frequencies f10, f20 and f30 of the imaging signal SB. Interference pattern images of the scattering tissue corresponding to the frequencies f10, f20 and f30 are mesh images I5, I6 and I7, respectively. An image I8 is formed by combining the images I5, I6 and I7.

As to delineation of a reflecting tissue of an examination object, the envelope of the time characteristics of the imaging signal SB shown in FIG. 9A is well shaped by Gaussian approximation, and also the S/N is good. Meanwhile, the envelope of the time characteristics of the imaging signal SA shown in FIG. 7A is non-Gaussian, and the bottom part is worse than that of the conventional imaging signal SB, but the shape of the envelope is impulsive, and the top part is sharp, so that ultrasound images become like stipple images. Further, the signal of the imaging signal SA is reduced as compared with that of the conventional imaging signal SB, so that the S/N decreases. Hence, it is preferable to use the imaging signal SA only for image regions where the S/N has a leeway, such as a shallow site.

As to delineation of a scattering tissue of an examination object, among the interference pattern images of the imaging signal SB shown in FIG. 9B, the interference pattern images where the scattering tissue is delineated, in the image I8 obtained by combining the images I5, I6 and I7, the lines of the image I6 are more strongly displayed than those of the images I5 and I7. Thus, influence of the center frequency (image I6) of the imaging frequency range is dominant in speckle granularity in an ultrasound image(s) of the imaging signal SB. Meanwhile, among the interference pattern images of the imaging signal SA shown in FIG. 7B, the interference pattern images where the scattering tissue is delineated, in the image I4 obtained by combining the images I1, I2 and I3, the lines of the images I1, I2 and I3 are displayed at the same strength. Thus, speckle granularity in an ultrasound image(s) of the imaging signal SA is high because, unlike the image I8 shown in FIG. 9B where influence of the interference pattern of the image I6 is dominant, different interference patterns are superimposed approximately evenly and hence the interference pitch is diversified. This allows the signal(s) derived from the scattering tissue at an ultra-shallow site or the like to be expressed in shades of the finely-granulated speckles, so that boundaries of tissues or the like, which have been difficult to recognize, can be visually confirmed.

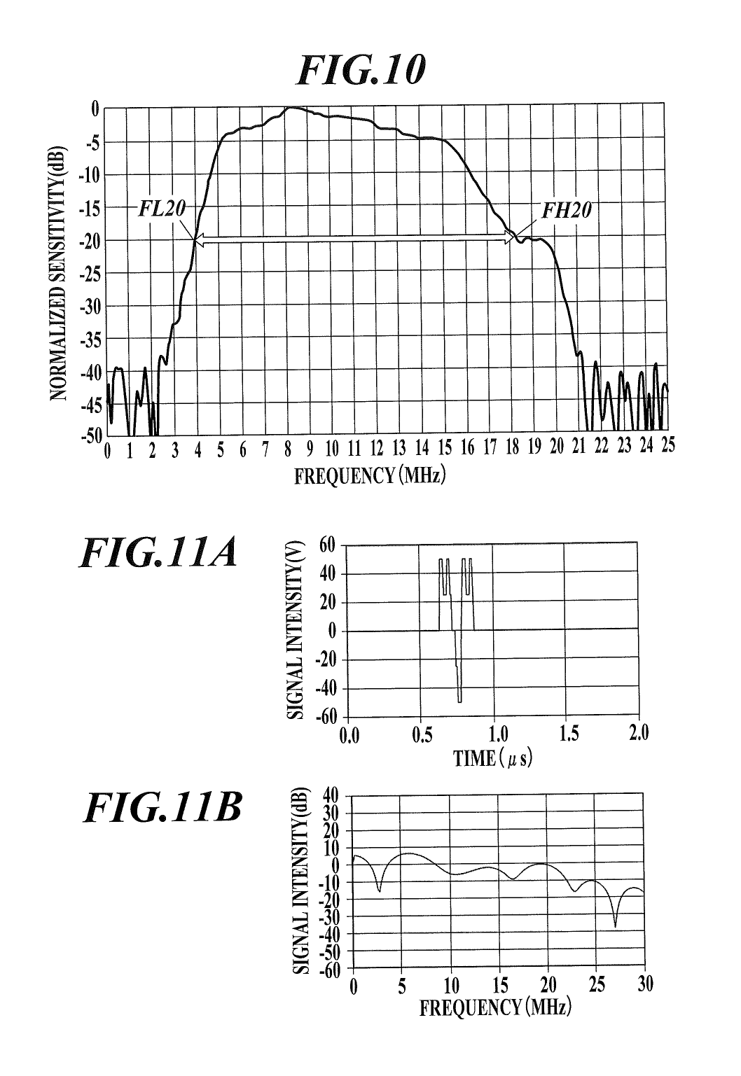

Next, with reference to FIG. 10 to FIG. 24, examples and comparative examples are described with specific examples of the ultrasound probe 2, the drive signal generated by the transmitter 12, the transmission ultrasound transmitted from the ultrasound probe 2 and the signal intensity correction filter of the filtering unit 14c of the ultrasound diagnostic apparatus S. As the examples and the comparative examples, Comparative Examples 1 and 2, Examples 1, 2, 3, 4, 5 and 6, Comparative Example 3 and Examples 7 and 8 are described in this order. Note that in all the comparative examples and the examples, the reception sound ray density was 0.075 mm.

### Comparative Example 1

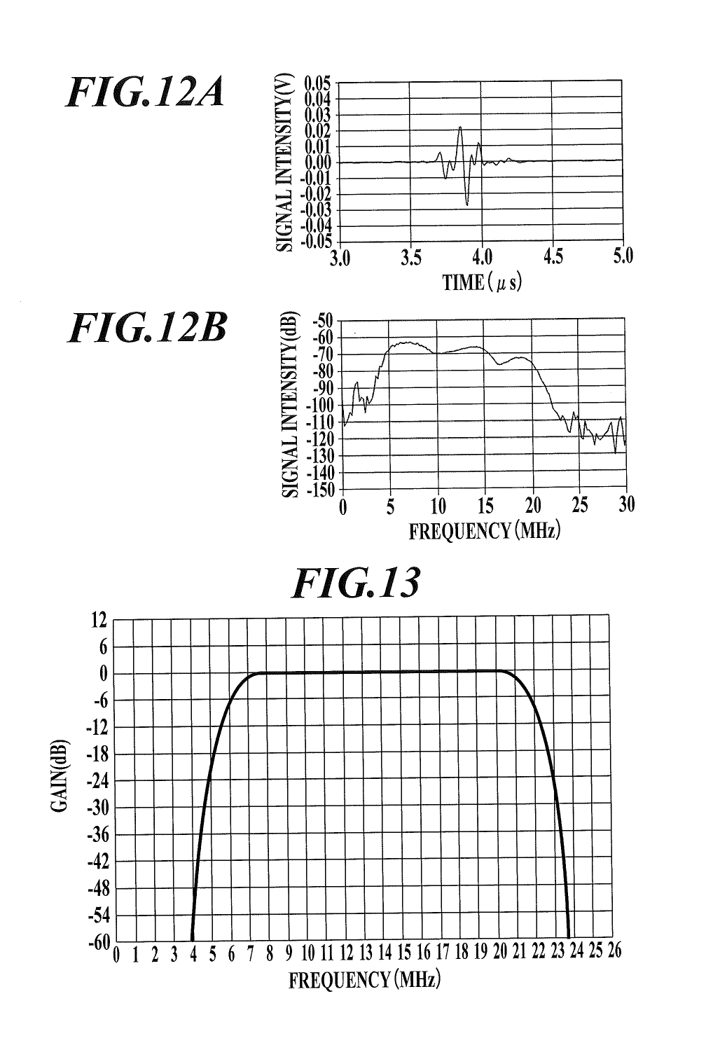

With reference to FIG. 10 to FIG. 13, Comparative Example 1 is described. FIG. 10 shows frequency characteristics of normalized sensitivity of an ultrasound probe P1. FIG. 11A shows time characteristics of signal intensity of a drive signal D1. FIG. 11B shows power spectrum of the drive signal D1. FIG. 12A shows time characteristics of signal intensity of transmission ultrasound U1. FIG. 12B shows power spectrum of the transmission ultrasound U1. FIG. 13 shows filter characteristics of a signal intensity correction filter FI1.

In Comparative Example 1, as the ultrasound probe 2, the ultrasound probe P1 having the frequency characteristics of the normalized sensitivity shown in FIG. 10 was used. The ultrasound probe P1 had a lower-end frequency FL20 of 3.9 MHz and an upper-end frequency FH20 of 18.2 MHz at −20 dB, thereby having a −20 dB fractional bandwidth of 129%, which is preferable because it is in a range of 120% or more.

The waveform of the drive signal D1 for Triad-THI generated by the transmitter 12 in Comparative Example 1 was the waveform of the time characteristics of the signal intensity [V] shown in FIG. 11A. The power spectrum obtained by Fourier transform on the time characteristics of the signal intensity [V] of the drive signal D1 shown in FIG. 11A had the frequency characteristics of the signal intensity [dB] shown in FIG. 11B. FIGS. 11A, 12A, 17A, 18A, 21A and 22A show time [ρs] on the horizontal axis and signal intensity (voltage) [V] on the vertical axis. FIGS. 11B, 12B, 17B, 18B, 21B and 22B show frequency [MHz] on the horizontal axis and signal intensity [dB] on the vertical axis.

The waveform of the transmission ultrasound U1 transmitted from the ultrasound probe P1 by input of the drive signal D1 shown in FIG. 11A to the ultrasound probe P1 in Comparative Example 1 was the waveform of the time characteristics of the signal intensity [V] shown in FIG. 12A. The power spectrum obtained by Fourier transform on the time characteristics of the signal intensity [V] of the transmission ultrasound U1 shown in FIG. 12A had the frequency characteristics of the signal intensity [dB] shown in FIG. 12B.

The signal intensity correction filter used in the filtering unit 14c in Comparative Example 1 was a non-adaptive signal intensity correction filter FI1 having the filter characteristics having the frequency characteristics of gain shown in FIG. 13. The signal intensity correction filter FI1 had the filter characteristics (gain) having a flat range as with the filter characteristics (signal passing degree or transmittance) of the signal intensity correction filter FIB shown in FIG. 8A.

Display conditions for ultrasound images generated by the ultrasound diagnostic apparatus S in Comparative Example 1 were a display depth [mm] of 10 mm and a display pixel resolution [mm/pixel] of 0.0189 mm/pixel.

The display pixel resolution in the embodiment is a numerical value indicating how many pixels are used to display the actual size in an echo image region. For example, if, in the echo image region, the length corresponding to 10 mm is displayed with 500 pixels, the display pixel resolution is 10 mm 500 pixels=0.02 mm/pixel. The display pixel resolution is changeable by an operator performing an operation to change the depth (Depth-change operation) to be displayed in the echo image region or an operation to enlarge a certain region (Zoom operation) with the operation inputter 11.

### Comparative Example 2

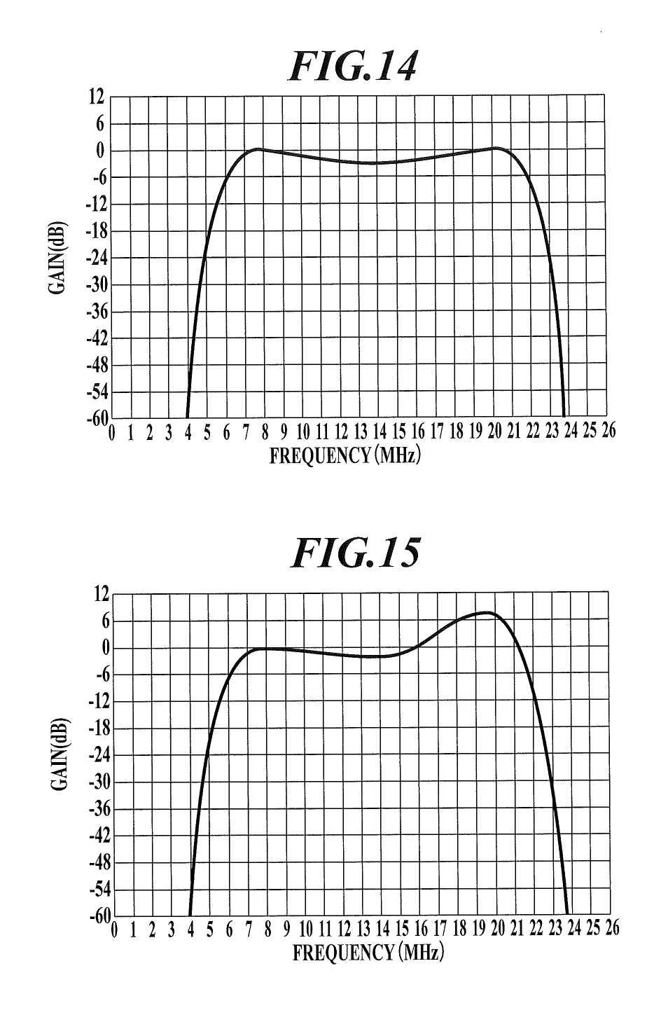

With reference to FIG. 14, Comparative Example 2 is described. FIG. 14 shows filter characteristics of a signal intensity correction filter FI2.

In Comparative Example 2, in the ultrasound diagnostic apparatus S, the ultrasound probe P1 was used as the ultrasound probe 2, the drive signal D1 was generated by the transmitter 12, and the transmission ultrasound U1 was transmitted. The signal intensity correction filter used in the filtering unit 14c in Comparative Example 2 was a non-adaptive signal intensity correction filter FI2 having the filter characteristics having the frequency characteristics of gain shown in FIG. 14. The signal intensity correction filter FI2 had non-flat filter characteristics (gain) as with the filter characteristics (signal passing degree or transmittance) of the signal intensity correction filter FIA shown in FIG. 6A. Display conditions for ultrasound images generated by the ultrasound diagnostic apparatus S in Comparative Example 2 were a display depth [mm] of 10 mm and a display pixel resolution [mm/pixel] of 0.0189 mm/pixel.

### Example 1

With reference to FIG. 15, Example 1 is described. FIG. 15 shows filter characteristics of a signal intensity correction filter FI3.

In Example 1, in the ultrasound diagnostic apparatus S, the ultrasound probe P1 was used as the ultrasound probe 2, the drive signal D1 was generated by the transmitter 12, and the transmission ultrasound U1 was transmitted. The signal intensity correction filter used in the filtering unit 14c in Example 1 was a non-adaptive signal intensity correction filter FI3 having the filter characteristics having the frequency characteristics of gain shown in FIG. 15. The signal intensity correction filter FI3 had non-flat filter characteristics (gain) as with the filter characteristics (signal passing degree or transmittance) of the signal intensity correction filter FIA shown in FIG. 6A. Display conditions for ultrasound images generated by the ultrasound diagnostic apparatus S in Example 1 were a display depth [mm] of 10 mm and a display pixel resolution [mm/pixel] of 0.0189 mm/pixel.

### Example 2

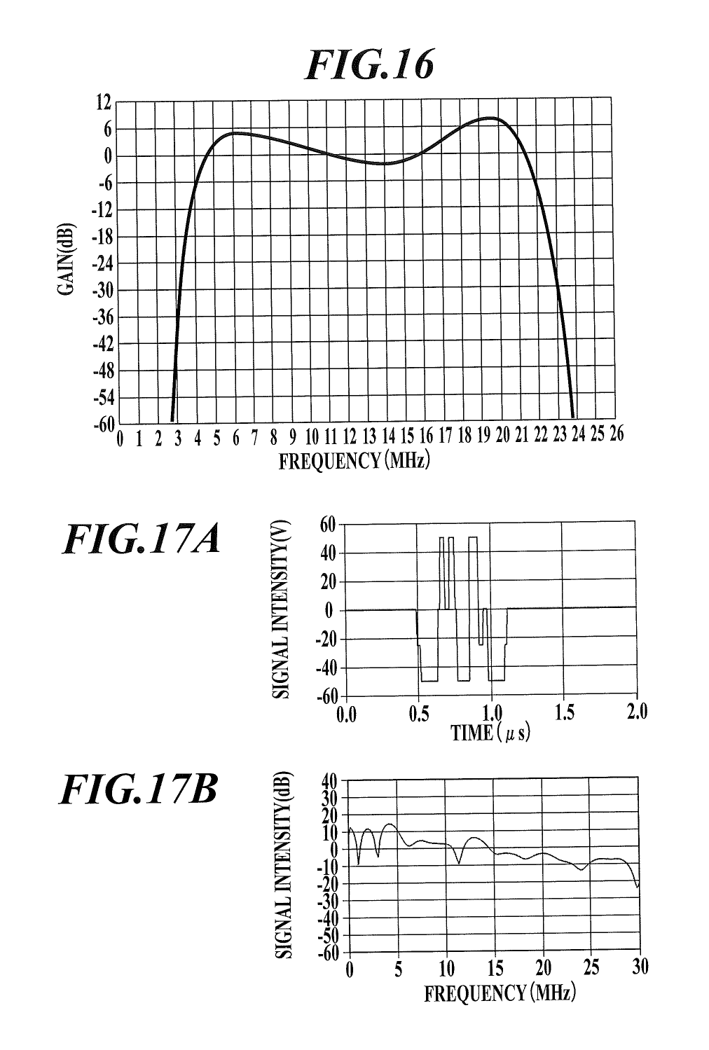

With reference to FIG. 16, Example 2 is described. FIG. 16 shows filter characteristics of a signal intensity correction filter FI4.

In Example 2, in the ultrasound diagnostic apparatus S, the ultrasound probe P1 was used as the ultrasound probe 2, the drive signal D1 was generated by the transmitter 12, and the transmission ultrasound U1 was transmitted. The signal intensity correction filter used in the filtering unit 14c in Example 2 was a non-adaptive signal intensity correction filter FI4 having the filter characteristics having the frequency characteristics of gain shown in FIG. 16. The signal intensity correction filter FI4 had non-flat filter characteristics (gain) as with the filter characteristics (signal passing degree or transmittance) of the signal intensity correction filter FIA shown in FIG. 6A. Display conditions for ultrasound images generated by the ultrasound diagnostic apparatus S in Example 2 were a display depth [mm] of 10 mm and a display pixel resolution [mm/pixel] of 0.0189 mm/pixel.

### Example 3

In Example 3, as with Example 2, in the ultrasound diagnostic apparatus S, the ultrasound probe P1 was used as the ultrasound probe 2, the drive signal D1 was generated by the transmitter 12, the transmission ultrasound U1 was transmitted, and the signal intensity correction filter FI4 was used in the filtering unit 14c. Display conditions for ultrasound images generated by the ultrasound diagnostic apparatus S in Example 3 were a display depth [mm] of 20 mm and a display pixel resolution [mm/pixel] of 0.0377 mm/pixel.

### Example 4

In Example 4, as with Example 2, in the ultrasound diagnostic apparatus S, the ultrasound probe P1 was used as the ultrasound probe 2, the drive signal D1 was generated by the transmitter 12, the transmission ultrasound U1 was transmitted, and the signal intensity correction filter FI4 was used in the filtering unit 14c. Display conditions for ultrasound images generated by the ultrasound diagnostic apparatus S in Example 4 were a display depth [mm] of 30 mm and a display pixel resolution [mm/pixel] of 0.0566 mm/pixel.

### Example 5

In Example 5, in the ultrasound diagnostic apparatus S, the ultrasound probe P1 was used as the ultrasound probe 2, the drive signal D1 was generated by the transmitter 12, and the transmission ultrasound U1 was transmitted. The signal intensity correction filter used in the filtering unit 14c in Example 5 was an adaptive signal intensity correction filter FI5. The signal intensity correction filter FI5 was an adaptive filter the coefficient of which was automatically determined by the filtering unit 14c to flatten the echo signal (sound ray data composed of extracted harmonic components) of the 7 to 20 MHz range, which was calculated by the frequency analyzer 14b as the flattening frequency range. Its frequency characteristics are not shown. Display conditions for ultrasound images generated by the ultrasound diagnostic apparatus S in Example 5 were a display depth [mm] of 10 mm and a display pixel resolution [mm/pixel] of 0.0189 mm/pixel.

### Example 6

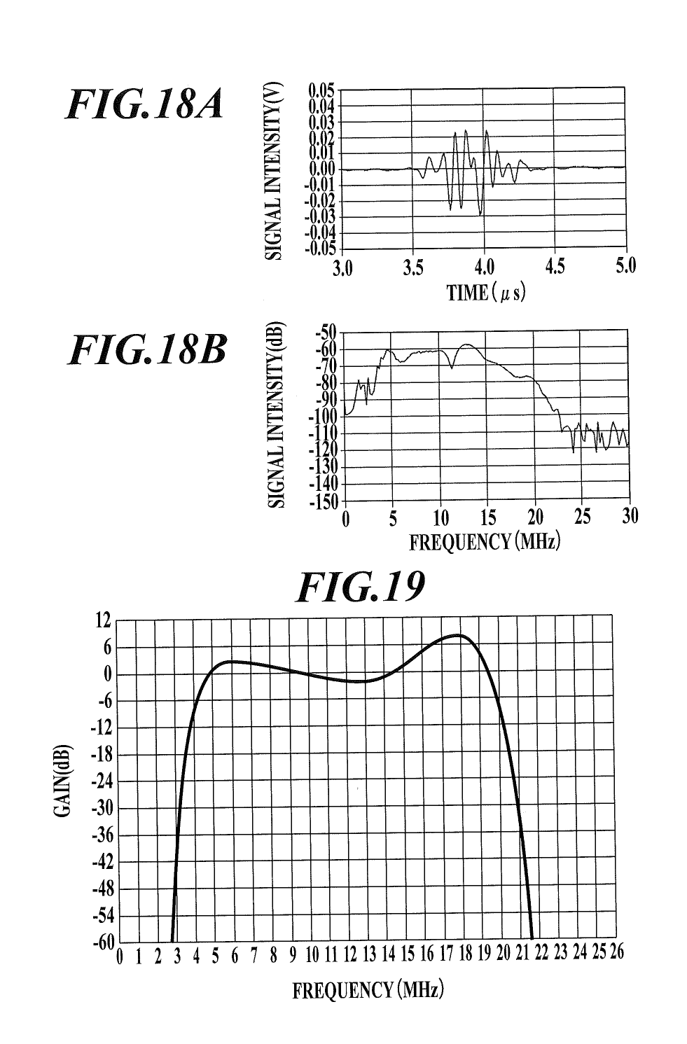

With reference to FIG. 17A to FIG. 19, Example 6 is described. FIG. 17A shows time characteristics of signal intensity of a drive signal D2. FIG. 17B shows power spectrum of the drive signal D2. FIG. 18A shows time characteristics of signal intensity of transmission ultrasound U2. FIG. 18B shows power spectrum of the transmission ultrasound U2. FIG. 19 shows filter characteristics of a signal intensity correction filter FI6.

In Example 6, in the ultrasound diagnostic apparatus S, the ultrasound probe P1 was used as the ultrasound probe 2. The waveform of the drive signal D2 for Triad-THI generated by the transmitter 12 in Example 6 was the waveform of the time characteristics of the signal intensity [V] shown in FIG. 17A. The power spectrum obtained by Fourier transform on the time characteristics of the signal intensity [V] of the drive signal D2 shown in FIG. 17A had the frequency characteristics of the signal intensity [dB] shown in FIG. 17B.

The waveform of the transmission ultrasound U2 transmitted from the ultrasound probe P1 by input of the drive signal D2 shown in FIG. 17A to the ultrasound probe P1 in Example 6 was the waveform of the time characteristics of the signal intensity [V] shown in FIG. 18A. The power spectrum obtained by Fourier transform on the time characteristics of the signal intensity [V] of the transmission ultrasound U2 shown in FIG. 18A had the frequency characteristics of the signal intensity [dB] shown in FIG. 18B.

The signal intensity correction filter used in the filtering unit 14c in Example 6 was a non-adaptive signal intensity correction filter FI6 having the filter characteristics having the frequency characteristics of gain shown in FIG. 19. The signal intensity correction filter FI6 had non-flat filter characteristics (gain) as with the filter characteristics (signal passing degree or transmittance) of the signal intensity correction filter FIA shown in FIG. 6A. Display conditions for ultrasound images generated by the ultrasound diagnostic apparatus S in Example 6 were a display depth [mm] of 10 mm and a display pixel resolution [mm/pixel] of 0.0189 mm/pixel.

### Comparative Example 3

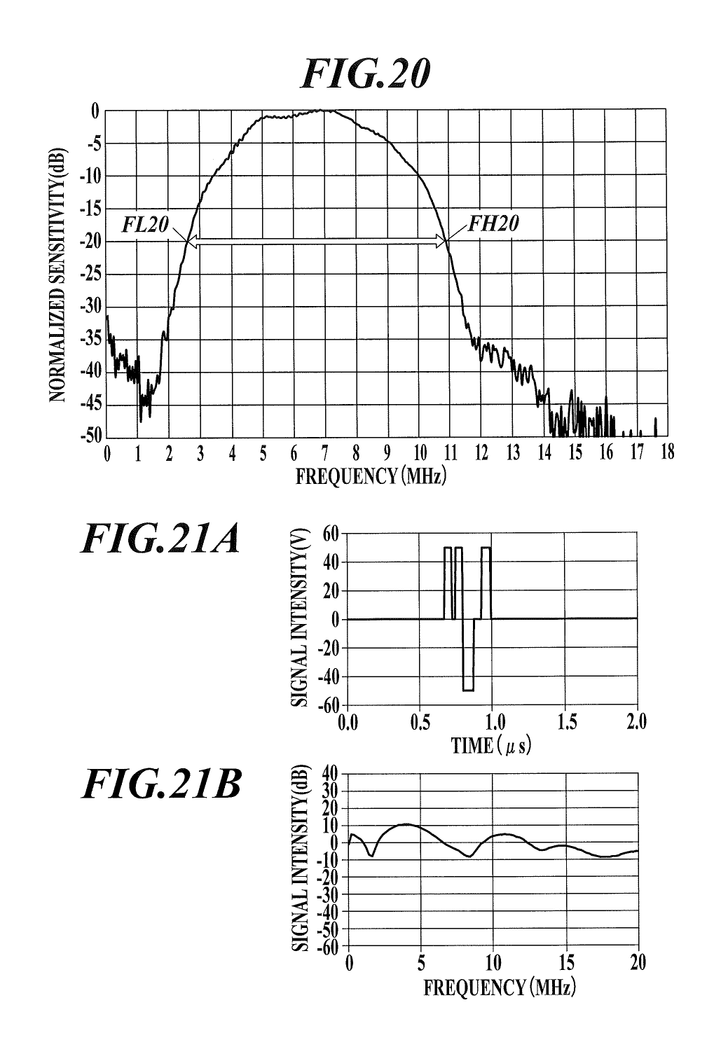

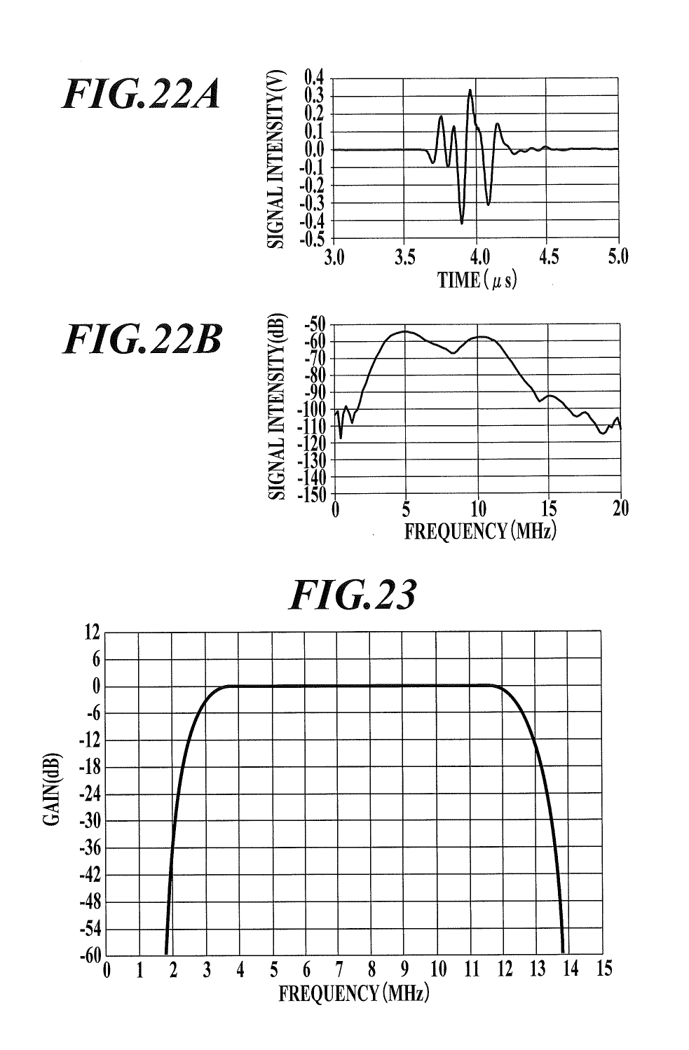

With reference to FIG. 20 to FIG. 23, Comparative Example 3 is described. FIG. 20 shows frequency characteristics of normalized sensitivity of an ultrasound probe P2. FIG. 21A shows time characteristics of signal intensity of a drive signal D3. FIG. 21B shows power spectrum of the drive signal D3. FIG. 22A shows time characteristics of signal intensity of transmission ultrasound U3. FIG. 22B shows power spectrum of the transmission ultrasound U3. FIG. 23 shows filter characteristics of a signal intensity correction filter FI7.

In Comparative Example 3, as the ultrasound probe 2, the ultrasound probe P2 having the frequency characteristics of the normalized sensitivity shown in FIG. 20 was used. The ultrasound probe P2 had a lower-end frequency FL20 of 2.6 MHz and an upper-end frequency FH20 of 10.9 MHz at −20 dB, thereby having a −20 dB fractional bandwidth of 123%, which is preferable because it is in a range of 120% or more.

The waveform of the drive signal D3 for Triad-THI generated by the transmitter 12 in Comparative Example 3 was the waveform of the time characteristics of the signal intensity [V] shown in FIG. 21A. The power spectrum obtained by Fourier transform on the time characteristics of the signal intensity [V] of the drive signal D3 shown in FIG. 21A had the frequency characteristics of the signal intensity [dB] shown in FIG. 21B.

The waveform of transmission ultrasound U3 transmitted from the ultrasound probe P2 by input of the drive signal D3 shown in FIG. 21A to the ultrasound probe P2 in Comparative Example 3 was the waveform of the time characteristics of the signal intensity [V] shown in FIG. 22A. The power spectrum obtained by Fourier transform on the time characteristics of the signal intensity [V] of the transmission ultrasound U3 shown in FIG. 22A had the frequency characteristics of the signal intensity [dB] shown in FIG. 22B.

The signal intensity correction filter used in the filtering unit 14c in Comparative Example 3 was a non-adaptive signal intensity correction filter FI7 having the filter characteristics having the frequency characteristics of gain shown in FIG. 23. The signal intensity correction filter FI7 had the filter characteristics (gain) having a flat range as with the filter characteristics (signal passing degree or transmittance) of the signal intensity correction filter FIB shown in FIG. 8A. Display conditions for ultrasound images generated by the ultrasound diagnostic apparatus S in Comparative Example 3 were a display depth [mm] of 10 mm and a display pixel resolution [mm/pixel] of 0.0189 mm/pixel.

### Example 7

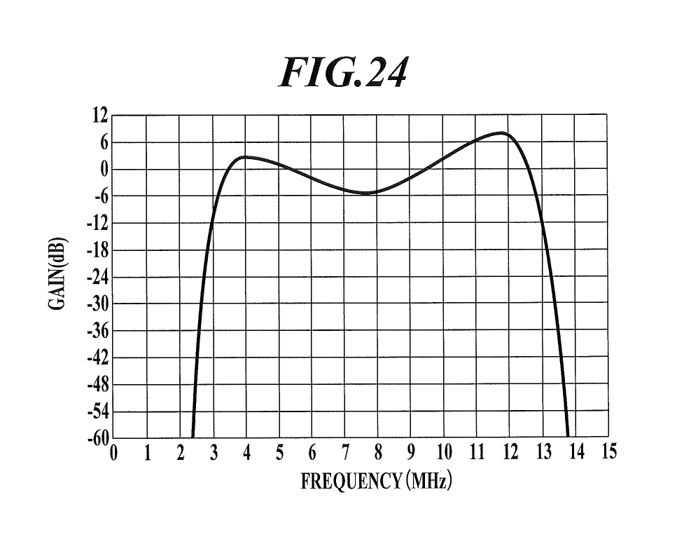

With reference to FIG. 24, Example 7 is described. FIG. 24 shows filter characteristics of a signal intensity correction filter FI8.

In Example 7, in the ultrasound diagnostic apparatus S, the ultrasound probe P2 was used as the ultrasound probe 2, the drive signal D3 was generated by the transmitter 12, and the transmission ultrasound U3 was transmitted. The signal intensity correction filter used in the filtering unit 14c in Example 7 was a non-adaptive signal intensity correction filter FI8 having the filter characteristics having the frequency characteristics of gain shown in FIG. 24. The signal intensity correction filter FI8 had non-flat filter characteristics (gain) as with the filter characteristics (signal passing degree or transmittance) of the signal intensity correction filter FIA shown in FIG. 6A. Display conditions for ultrasound images generated by the ultrasound diagnostic apparatus S in Example 7 were a display depth [mm] of 10 mm and a display pixel resolution [mm/pixel] of 0.0189 mm/pixel.

### Example 8

In Example 8, as with Example 7, in the ultrasound diagnostic apparatus S, the ultrasound probe P2 was used as the ultrasound probe 2, the drive signal D3 was generated by the transmitter 12, the transmission ultrasound U3 was transmitted, and the signal intensity correction filter FI8 was used in the filtering unit 14c. Display conditions for ultrasound images generated by the ultrasound diagnostic apparatus S in Example 8 were a display depth [mm] of 20 mm and a display pixel resolution [mm/pixel] of 0.0377 mm/pixel.

In the ultrasound diagnostic apparatus S, with the above-described ultrasound probes, drive signals, transmission ultrasounds, display depths and display pixel resolutions in Comparative Examples 1 to 3 and Examples 1 to 8, ultrasound image data of examination objects were generated and displayed. These imaging conditions are shown in TABLE 1 below.

TABLE 1

| | IMAGING CONDITIONS | | | | | | | |
|---|---|---|---|---|---|---|---|---|
| | | TRANSMISSION | | | IMAGING | | | FLATTENING |
| | | WAVEFORMS | | | FREQUENCY | | | FREQUENCY |
| | ULTRA- | DRIVE | TRANSMISSION | FILTER | RANGE (MHz) | | | RANGE (MHz) |
| | SOUND | SIGNAL | ULTRASOUND | CHARACTER- | LOWER | UPPER | | LOWER |
| | PROBE | WAVEFORM | WAVEFORM | ISTICS | END | END | WIDTH | END |
| COMPARATIVE | P1 | D1 | U1 | FI1 | 7.5 | 22.0 | 14.5 | 9.4 |
| EXAMPLE 1 | | | | | | | | |
| COMPARATIVE | | | | FI2 | 7.5 | 22.0 | 14.5 | 9.0 |
| EXAMPLE 2 | | | | | | | | |
| EXAMPLE 1 | | | | FI3 | 7.5 | 22.0 | 14.5 | 9.0 |
| EXAMPLE 2 | | | | FI4 | 6.0 | 22.0 | 16.0 | 7.0 |
| EXAMPLE 3 | | | | FI4 | 6.0 | 22.0 | 16.0 | 7.0 |
| EXAMPLE 4 | | | | FI4 | 6.0 | 22.0 | 16.0 | 7.0 |
| EXAMPLE 5 | | | | FI5 | 6.0 | 22.0 | 16.0 | 7.0 |
| EXAMPLE 6 | | D2 | U2 | FI6 | 5.0 | 19.5 | 14.5 | 6.3 |
| COMPARATIVE | P2 | D3 | U3 | FI7 | 3.5 | 13.0 | 9.5 | 6.0 |
| EXAMPLE 3 | | | | | | | | |
| EXAMPLE 7 | | | | FI8 | 3.5 | 13.0 | 9.5 | 4.0 |
| EXAMPLE 8 | | | | FI8 | 3.5 | 13.0 | 9.5 | 4.0 |
| | IMAGING CONDITIONS | | | | | | | |
| | FLATTENING FREQUENCY | | | | IN-VIVO | DISPLAY CONDITIONS | | |
| | RANGE (MHz) | | | | WAVELENGTH | | | IN-VIVO |
| | | | UPPER | | CORRESPONDING | | DISPLAY | WAVELENGTH |
| | | | END TO | | TO UPPER END | DIS- | PIXEL | TO DISPLAY |
| | | | LOWER | FLAT- | OF FLATTENING | PLAY | RESOLU- | PIXEL |
| | UPPER | | END | NESS | FREQUENCY | DEPTH | TION | RESOLUTION |
| | END | WIDTH | RATIO | (%) | RANGE (mm) | (mm) | (mm/Pixel) | RATIO |
| COMPARATIVE | 13.6 | 4.2 | 1.4 | 29 | 0.113 | 10 | 0.0189 | 5.96 |
| EXAMPLE 1 | | | | | | | | |
| COMPARATIVE | 17.0 | 8.0 | 1.9 | 55 | 0.090 | 10 | 0.0189 | 4.77 |
| EXAMPLE 2 | | | | | | | | |
| EXAMPLE 1 | 20.0 | 11.0 | 2.2 | 76 | 0.077 | 10 | 0.0189 | 4.05 |
| EXAMPLE 2 | 20.0 | 13.0 | 2.9 | 81 | 0.077 | 10 | 0.0189 | 4.05 |
| EXAMPLE 3 | 20.0 | 13.0 | 2.9 | 81 | 0.077 | 20 | 0.0377 | 2.03 |
| EXAMPLE 4 | 20.0 | 13.0 | 2.9 | 81 | 0.077 | 30 | 0.0566 | 1.35 |
| EXAMPLE 5 | 20.0 | 13.0 | 2.9 | 81 | 0.077 | 10 | 0.0189 | 4.05 |
| EXAMPLE 6 | 18.0 | 11.7 | 2.9 | 81 | 0.085 | 10 | 0.0189 | 4.51 |
| COMPARATIVE | 9.2 | 3.2 | 1.5 | 34 | 0.166 | 10 | 0.0189 | 8.81 |
| EXAMPLE 3 | | | | | | | | |
| EXAMPLE 7 | 11.6 | 7.6 | 2.9 | 80 | 0.132 | 10 | 0.0189 | 6.99 |
| EXAMPLE 8 | 11.6 | 7.6 | 2.9 | 80 | 0.132 | 20 | 0.0377 | 3.50 |

The “Imaging Frequency Range” in TABLE 1 indicates an imaging frequency range [MHz] in an imaging signal obtained by filtering in the filtering unit 14c, and the upper-end and lower-end frequencies [MHz] of the imaging frequency range and the value [MHz] of the width between the upper-end frequency and the lower-end frequency are shown therein. The “Flattening Frequency Range” in TABLE 1 indicates a frequency range having signal intensities of not below −3 dB from the maximum intensity at the frequencies in an imaging signal obtained by filtering in delineating a tissue part between an index finger and a middle finger of an examination object, and the upper-end and lower-end frequencies [MHz] of the flattening frequency range, the value [MHz] of the width between the upper-end frequency and the lower-end frequency, and the upper-end-frequency-to-lower-end-frequency ratio (=upper-end frequency of flattening frequency range/lower-end frequency of flattening frequency range) are shown therein.

The “Flatness [%]” in TABLE 1 indicates a value of (width of flattening frequency range)/(width of imaging frequency range). About the flattening frequency range, an in-vivo wavelength corresponding to the upper-end frequency of the flattening frequency range was calculated (i.e. the upper-end frequency of the flattening frequency range was converted into a wavelength in a living body) and is shown as the “In-vivo Wavelength Corresponding to Upper End of Flattening Frequency Range [mm]” in TABLE 1. As the “Display Conditions” in TABLE 1, in addition to the display depth and the display pixel resolution, the in-vivo-wavelength-to-display-pixel-resolution ratio (=in-vivo wavelength (calculated above)/display pixel resolution) was calculated and is shown as the “In-Vivo Wavelength to Display Pixel Resolution Ratio” in TABLE 1.

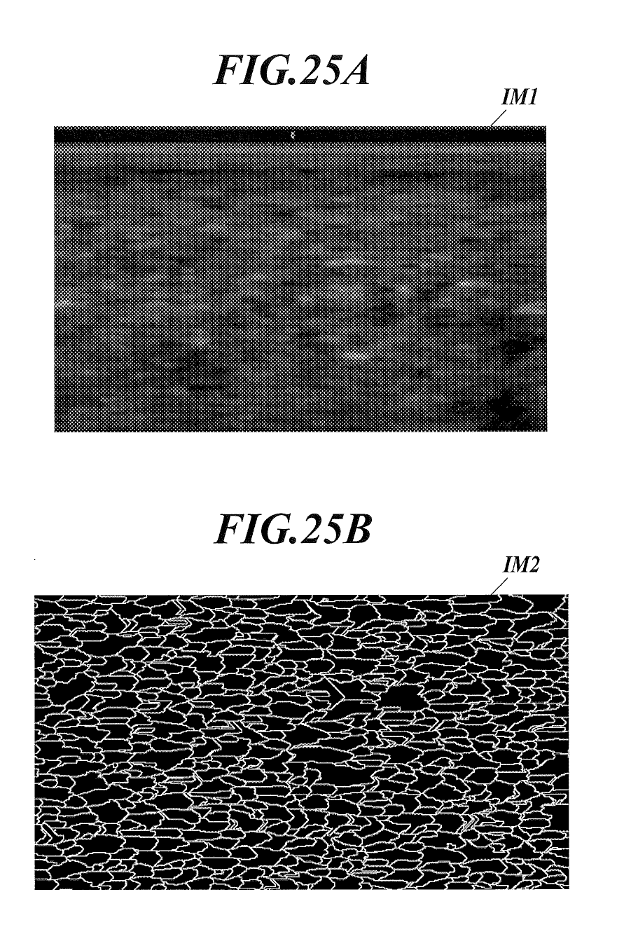

With reference to FIG. 25A and FIG. 25B, image quality evaluation of the ultrasound images generated under the imaging conditions of Comparative Examples 1 to 3 and Examples 1 to 8 is described. FIG. 25A shows a B-mode image IM1. FIG. 25B shows an image IM2 obtained by watershed segmentation on the B-mode image IM1.

In the ultrasound diagnostic apparatus S, the ultrasound images were generated under the imaging conditions of Comparative Examples 1 to 3 and Examples 1 to 8 and displayed on the display 17. The image qualities of the displayed ultrasound images were evaluated. The results of the image quality evaluation are shown in TABLE 2 below.

TABLE 2

| | IMAGE QUALITY EVALUATION | | | | |
|---|---|---|---|---|---|
| | | CLINICAL IMAGE EVALUATION | | | |
| | | MEDIAN NERVE | | ADHERENT | |
| | SPECKLE | TO TERMINAL | FLEXOR | PART OF | |
| | GRANULARITY | OF MIDDLE FINGER | TENDON AT | VOLAR PLATE | TOTAL |
| | (mm²) | ON PALMAR SIDE | MP JOINT | OF MP JOINT | SCORE |
| COMPARATIVE | 0.040 | 3.8 | 5.4 | 4.0 | 13.2 |
| EXAMPLE 1 | | | | | |
| COMPARATIVE | 0.038 | 4.2 | 5.3 | 4.7 | 14.2 |
| EXAMPLE 2 | | | | | |
| EXAMPLE 1 | 0.022 | 9.0 | 8.2 | 8.3 | 25.5 |
| EXAMPLE 2 | 0.020 | 9.1 | 8.0 | 8.6 | 25.7 |
| EXAMPLE 3 | 0.024 | 8.0 | 7.2 | 7.6 | 22.8 |
| EXAMPLE 4 | 0.028 | 7.1 | 6.6 | 6.8 | 20.5 |
| EXAMPLE 5 | 0.020 | 9.1 | 9.0 | 9.1 | 27.2 |
| EXAMPLE 6 | 0.024 | 8.6 | 7.8 | 8.0 | 24.4 |
| COMPARATIVE | 0.053 | 2.9 | 4.9 | 3.9 | 11.7 |
| EXAMPLE 3 | | | | | |
| EXAMPLE 7 | 0.026 | 6.9 | 7.0 | 7.0 | 20.9 |
| EXAMPLE 8 | 0.030 | 6.5 | 6.3 | 6.3 | 19.1 |

The “Speckle Granularity [mm²]” in TABLE 2 indicates the mean area [mm²] of cells obtained by watershed segmentation on an image of the tissue part between the index finger and the middle finger (using ImageJ V1.49 and Watershed Algorithm.jar). Watershed segmentation is a method of likening brightness gradients of an image to mountains and valleys and segmenting rivers running through the valleys by regions enclosed by the mountains. For example, when watershed segmentation is performed on the B-mode image IM1 shown in FIG. 25A, the image IM2 shown in FIG. 25B is obtained. The mean area [mm²] of the cells enclosed by white lines in the image IM2 is regarded as speckle granularity [mm²].

Examination objects subjected to the clinical image evaluation were the median nerve to the terminal of the middle finger on the palmar side, the flexor tendon at an MP joint and an adherent part(s) of the volar plate of the MP joint of a living body as shown in TABLE 2. The score for each examination object in the clinical image evaluation was determined as follows: obtained grades from 10 peoples in total who were doctors engaging in orthopedics or the like and medical technologists, with evaluation criteria below; and averaged the obtained grades (rounded off to the whole number). Thus, the delineation score for each examination object was obtained. The evaluation criteria are as follows: 10=excellent delineation to recognize the tissue condition; 8=delineation with no problem in practical use to recognize the tissue condition; 6=delineation that is not satisfactory but is enough to recognize the tissue condition; 4=delineation with a difficulty to recognize the tissue condition; and 2=delineation that is too low to recognize the tissue condition.

The “Total Score” under the “Clinical Image Evaluation” in TABLE 2 indicates the total of the scores about delineation of the median nerve to the terminal of the middle finger on the palmar side, the flexor tendon at the MP joint and the adherent part(s) of the volar plate of the MP joint.

As shown in TABLE 2, preferably, the upper-end-frequency-to-lower-end-frequency ratio of the flattening frequency range is 2.0 or more (Examples 1 to 8) because this achieves high speckle granularity (i.e. a small value of speckle granularity) and a high total score.

When the upper-end-frequency-to-lower-end-frequency ratio is 2.0 or more, speckle patterns having respective repeating units that are twice or more different from each other are superimposed at uniform intensity, so that the speckle pattern corresponding to the lower-end frequency is divided into four or more, and the fine granulation effect increases accordingly. There is no upper limit in the upper-end-frequency-to-lower-end-frequency ratio, but if the ratio is set to an unnecessarily large value, the disadvantageous effect due to the decrease in the S/N exceeds the advantageous effect due to the fine granulation. Hence, preferably, the upper limit thereof is arbitrarily set within a range in which the advantageous effect exceeds the disadvantageous effect.

The frequency range to which the embodiment is applied is not limited, and the effect(s) can be obtained in any frequency range. However, as shown in TABLE 2, preferably, the upper-end frequency of the flattening frequency range is 20 MHz or more (Examples 1 to 5) because this achieves higher speckle granularity (i.e. a smaller value of speckle granularity) and a higher total score, and can exhibit a high effect of visualization of the tissues, which have been difficult to observe, and have a high degree of usefulness.

Further, as shown in TABLE 2, preferably, the flatness based on the imaging frequency range and the flattening frequency range is 80% or more (Examples 2 to 8) because this achieves higher speckle granularity and a higher total score.

Further, preferably, the in-vivo-wavelength-to-display-pixel-resolution ratio is 4.0 or more. This is because, as shown in TABLE 2, among Examples 2, 3, 4, 7 and 8, when the in-vivo-wavelength-to-display-pixel-resolution ratio is 4.0 or more (Examples 2 and 7), the display pixel resolution is suitable for observation of small tissues, and higher speckle granularity and a higher total score are achieved.

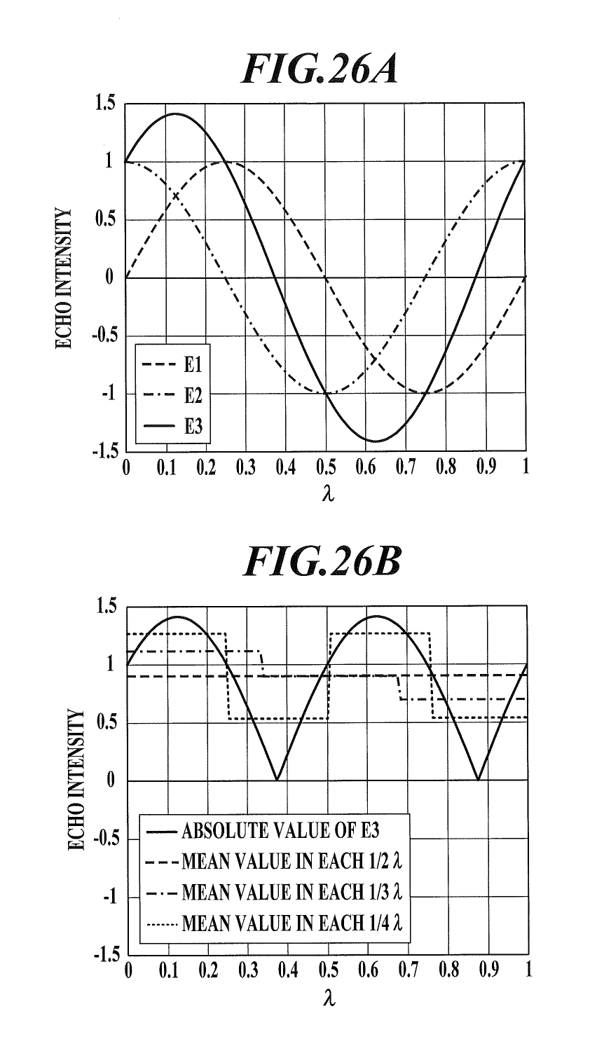

Here, with reference to FIG. 26A and FIG. 26B, the reason why the in-vivo-wavelength-to-display-pixel-resolution ratio, which is relevant to the flattening frequency range, is preferably 4.0 or more is described. FIG. 26A shows wavelength characteristics of echo intensity of a combined echo signal E3 of echo signals E1 and E2. FIG. 26B shows wavelength characteristics of echo intensity of the absolute value and the mean values in divided wavelength of the combined echo signal E3. FIG. 26A shows, on the vertical axis, echo intensity in relation to the intensity at the maximum amplitude (maximum amplitude intensity) of each of the echo signals E1 and E2 being 1, and, on the horizontal axis, values in relation to wavelength λ of the echo signal E1 being 1. The vertical axis and the horizontal axis in FIG. 26B correspond to the vertical axis and the horizontal axis in FIG. 26A, respectively.

If an echo scattering source is present at a point shorter than the wavelength; for example, if the echo signal E1 and the echo signal E2 are combined within the wavelength as shown in FIG. 26A, the combined echo signal E3 is as shown in FIG. 26A. In an echo image, amplitude intensity acts as image brightness, so that the absolute value of the combined echo signal E3 shown in FIG. 26B is the original information of image information. When this is expressed by ½λ, it is expressed by the mean value in the 0λ to 0.5λ range and the mean value in the 0.5λ to 1λ range, and no difference in image signal intensity, namely, display brightness, is generated between these two. In the case of ⅓λ, some difference in display brightness is generated therebetween, but it is not enough. In the case of ¼λ or more finely dividing λ, difference in brightness close to that of the original information can be displayed. Although the above is an example and the combined state of multiple signals is various, the in-vivo-wavelength-to-display-pixel-resolution ratio is preferably 4.0 or more because if λ is divided by less than four, namely, if the above ratio is less than 4.0, the information may be lost at the stage of displaying.

Preferably, the controller 18 adjusts the display size of the ultrasound image data to be displayed on the screen of the display 17 automatically or in response to input of the display size or the like by an operator with the operation inputter 11 such that the in-vivo-wavelength-to-display-pixel-resolution ratio is 4.0 or more.

Further, as shown in TABLE 2, Example 5, in which an adaptive signal intensity correction filter was used, obtained the effect(s) due to flattening stably, regardless of scattering characteristics of the delineated tissues, as compared with Example 2, in which a non-adaptive signal intensity correction filter was used, and Example 5 achieved speckle granularity as high as that of Example 2. Thus, preferably, an adaptive signal intensity correction filter is used because this achieves, in addition to high speckle granularity, a high score about any site and a high total score accordingly.

Note that the reception sound ray density, which affects information density in the orientation direction of the ultrasound probe 2, is preferably equivalent to the in-vivo wavelength corresponding to the upper-end frequency of the flattening frequency range or less. That is, if the upper-end frequency of the flattening frequency range is 20 MHz, the in-vivo wavelength corresponding to the upper-end frequency of the flattening frequency range is 0.0765 mm, and hence the receiving interval is preferably equal to or less than this numerical value. In all the above comparative examples and examples, the reception sound ray density was 0.075 mm, which satisfies the above preferable condition for the reception sound ray density. This strikes a balance between information density in the distance direction and that in the orientation direction, and produces speckle granules having a small aspect ratio.

As described above, according to the embodiment, the ultrasound diagnostic apparatus S includes: the transmitter 12 that generates a drive signal and outputs the drive signal to the ultrasound probe 2 to cause the ultrasound probe 2 to generate transmission ultrasound; the receiver 13 that receives an echo signal from the ultrasound probe 2; the filtering unit 14c that adjusts signal intensity of the echo signal to signal intensity having a flat frequency range; and the envelope detector 14d that generates ultrasound image data from the echo signal having the adjusted signal intensity (i.e. an imaging signal).

This achieves high speckle granularity and a high total score, and can granulate speckles as small/fine as possible, the speckles being generated by interference of scattered echo signals, and extract microstructures with the speckles as the minimum units of significant image information.

In particular, in the field of orthopedics/anesthesia, this, for example, can visualize a terminal nerve bundle(s), which has been difficult to follow to its utmost terminal because it becomes smaller by branching from the proximal point to the distal point, and also can clearly delineate tissue at the boundary of an adherent part(s) of a volar plate(s), and thereby makes diagnosis of microstructures and so forth possible. Further, in the field of dermatology, in melanoma thickness observation to judge whether to perform biopsy of lymph nodes on the basis of whether the thickness of a tumor is at least 1 mm, the present invention is expected to be utilized for distinguishing a melanoma and a melanocyte, which has been difficult, and for distinguishing a melanoma and a pseudohorn cyst, which are resemble in appearance when observed with eyes but different in pathological configuration and in benignancy/malignancy.

Further, the filtering unit 14c adjusts the signal intensity of the echo signal such that the upper-end-frequency-to-lower-end-frequency ratio of the flat frequency range is 2.0 or more. This achieves higher speckle granularity and a higher total score, and can exhibit a high effect of visualization of tissues, which have been difficult to observe, and have a high degree of usefulness.

Further, the filtering unit 14c adjusts the signal intensity of the echo signal such that the upper-end frequency of the flat frequency range is 20 MHz or more. This achieves higher speckle granularity and a higher total score, and can exhibit a high effect of visualization of tissues, which have been difficult to observe, and have a high degree of usefulness.

Further, the filtering unit 14c adjusts the signal intensity of the echo signal such that the flatness of the echo signal is 80% or more. This achieves higher speckle granularity and a higher total score, and can granulate speckles generated by interference of scattered echo signals to be even smaller/finer, and extract microstructures with the speckles as the minimum units of significant image information.

Further, the ultrasound diagnostic apparatus S further includes the controller 18 that displays the ultrasound image data on the display 17 at a display pixel resolution. The controller 18 adjusts the signal intensity of the echo signal or the display pixel resolution such that the in-vivo-wavelength-to-display-pixel-resolution ratio, which is relevant to the flat frequency range, is 4.0 or more.

This achieves higher speckle granularity and a higher total score, and can granulate speckles generated by interference of scattered echo signals to be even smaller/finer, and extract microstructures with the speckles as the minimum units of significant image information.

Further, the filtering unit 14c adjusts the signal intensity of the echo signal by filtering the echo signal with a signal intensity correction filter. This can easily adjust the signal intensity of an echo signal(s).

Further, the ultrasound diagnostic apparatus S further includes the frequency analyzer 14b that performs frequency analysis on the signal intensity in the flat frequency range of the echo signal and generates an analysis result. The filtering unit 14c sets the coefficient of an adaptive signal intensity correction filter according to the analysis result and uses the filter for the filtering. This can appropriately flatten an echo signal(s) according to the signal intensity of the echo signal, achieves higher speckle granularity and a higher total score, and can granulate speckles generated by interference of scattered echo signals to be even smaller/finer, and extract microstructures with the speckles as the minimum units of significant image information.

Further, the transmitter 12 generates a drive signal for THI. The ultrasound diagnostic apparatus S further includes the harmonic component extractor 14a that extracts a harmonic component(s) of the received echo signal. The filtering unit 14c adjusts the signal intensity of an echo signal of the extracted harmonic component. This can generate excellent ultrasound images with sidelobe artifacts reduced, on the basis of harmonic components.

Further, in THI, as described in Japanese Patent Application Publication No. 2014-168555, preferably, the frequency power spectrum of a transmission pulse signal as the drive signal has intensity peaks in a frequency band included in a transmission frequency band at −20 dB of the ultrasound probe 2 on the lower frequency side and the higher frequency side than the center frequency of the transmission frequency band, respectively, and the intensity in a frequency range between the intensity peaks is −20 dB or more with the maximum value of the intensities of the intensity peaks being a reference. This can obtain broadband harmonic components in a wide depth range, generate more excellent ultrasound images with sidelobe artifacts reduced, and eliminate a need to add an intricate circuit to form/shape the waveform of a pulse signal(s), and thereby can maintain high resolution about transmission ultrasound while reducing cost. Further, according to ultrasound of fundamentals, the waveform of the ultrasound with a large amplitude and a short pulse can be obtained. This can maintain high resolution while improving penetration (invasion depth) by increasing low frequency components.

Those described in the above embodiment and the like are preferred examples of the ultrasound diagnostic apparatus and the image forming method of the present invention, and not intended to limit the present invention.

For example, in the above embodiment, the ultrasound diagnostic apparatus S is configured to transmit and receive ultrasound and generate ultrasound images by Triad-THI, which is the broadband transmission/reception method described in Japanese Patent Application Publication Nos. 2014-168555 and 2016-214622, but not limited thereto, and hence may be configured to transmit and receive ultrasound and generate ultrasound images by another THI, or may be configured to transmit and receive ultrasound and generate ultrasound images in an ordinary way, without extracting harmonic components.

Further, detailed configurations and detailed actions of the devices or the like of the ultrasound diagnosis apparatus S in the above embodiment and the like can also be appropriately modified without departing from the spirit of the present invention.

Although embodiments of the present invention have been described and illustrated in detail, it is clearly understood that the same is by way of illustration and example only and is not to be taken by way of limitation, and the scope of the present invention should be interpreted by terms of the appended claims.

What is claimed is:

1. An ultrasound diagnostic apparatus comprising:
an ultrasound probe that transmits transmission ultrasound to an examination object, receives echo from the examination object and generates an echo signal;
a transmitter that generates a drive signal and outputs the drive signal to the ultrasound probe to cause the ultrasound probe to generate the transmission ultrasound;
a receiver that receives the echo signal from the ultrasound probe;
a signal intensity adjuster that adjusts signal intensity of the echo signal to signal intensity having a flat frequency range; and
an image data generator that generates ultrasound image data from the echo signal having the adjusted signal intensity.

2. The ultrasound diagnostic apparatus according to claim 1, wherein the signal intensity adjuster adjusts the signal intensity of the echo signal such that a ratio obtained by dividing an upper-end frequency of the flat frequency range by a lower-end frequency of the flat frequency range is 2.0 or more.

3. The ultrasound diagnostic apparatus according to claim 1, wherein the signal intensity adjuster adjusts the signal intensity of the echo signal such that an upper-end frequency of the flat frequency range is 20 MHz or more.

4. The ultrasound diagnostic apparatus according to claim 1, wherein the signal intensity adjuster adjusts the signal intensity of the echo signal such that a flatness obtained by dividing the flat frequency range by an imaging frequency range is 80% or more.

5. The ultrasound diagnostic apparatus according to claim 1, further comprising a hardware processor that displays the ultrasound image data on a display at a display pixel resolution, wherein
the hardware processor adjusts a display size on a screen of the display such that a ratio obtained by dividing an in-vivo wavelength corresponding to an upper-end frequency of the flat frequency range by the display pixel resolution is 4.0 or more.

6. The ultrasound diagnostic apparatus according to claim 1, wherein the signal intensity adjuster adjusts the signal intensity of the echo signal by filtering the echo signal with a signal intensity correction filter.

7. The ultrasound diagnostic apparatus according to claim 6, further comprising a frequency analyzer that performs frequency analysis on the signal intensity in the flat frequency range of the echo signal and generates an analysis result, wherein
the signal intensity correction filter is an adaptive signal intensity correction filter, and
the signal intensity adjuster sets a coefficient of the adaptive signal intensity correction filter according to the analysis result and uses the adaptive signal intensity correction filter for the filtering.

8. The ultrasound diagnostic apparatus according to claim 1, wherein
the transmitter generates the drive signal for tissue harmonic imaging,
the ultrasound diagnostic apparatus further comprises a harmonic component extractor that extracts a harmonic component of the received echo signal, and
the signal intensity adjuster adjusts signal intensity of an echo signal of the extracted harmonic component.

9. The ultrasound diagnostic apparatus according to claim 8, wherein
in the tissue harmonic imaging,
a frequency power spectrum of a transmission pulse signal as the drive signal has intensity peaks in a frequency band included in a transmission frequency band at −20 dB of the ultrasound probe on a lower frequency side and a higher frequency side than a center frequency of the transmission frequency band, respectively, and
an intensity in a frequency range between the intensity peaks is −20 dB or more with a maximum value of intensities of the intensity peaks being a reference.

10. An image forming method comprising:
adjusting signal intensity of an echo signal received from an ultrasound probe that transmits transmission ultrasound to an examination object and receives echo from the examination object to signal intensity having a flat frequency range; and
generating ultrasound image data from the echo signal having the adjusted signal intensity.
